SEQUENCE LISTING (26 sequences)

SEQ ID NO: 1 - protein, 9 aa, Artificial Sequence
  An artificially synthesized peptide
RLLVPTQFV

SEQ ID NO: 2 - protein, 9 aa, Artificial Sequence
  An artificially synthesized peptide
KILAHNNFV

SEQ ID NO: 3 - protein, 9 aa, Artificial Sequence
  An artificially synthesized peptide
KIQEILTQV

SEQ ID NO: 4 - protein, 9 aa, Artificial Sequence
  An artificially synthesized peptide
LQWEVLDSL

SEQ ID NO: 5 - protein, 9 aa, Artificial Sequence
  An artificially synthesized peptide
KIPVSGPFL

SEQ ID NO: 6 - protein, 9 aa, Artificial Sequence
  An artificially synthesized peptide
NLSSAEVVV

SEQ ID NO: 7 - protein, 9 aa, Artificial Sequence
  An artificially synthesized peptide
KQTQSKIDV

SEQ ID NO: 8 - protein, 9 aa, Artificial Sequence
  An artificially synthesized peptide
GLNLNALGL

SEQ ID NO: 9 - protein, 9 aa, Artificial Sequence
  An artificially synthesized peptide
SLLVQYGVV

SEQ ID NO: 10 - protein, 9 aa, Artificial Sequence
  An artificially synthesized peptide
GLFPPTSGM

SEQ ID NO: 11 - protein, 9 aa, Artificial Sequence
  An artificially synthesized peptide
KQGQHIKQL

SEQ ID NO: 12 - protein, 9 aa, Artificial Sequence
  An artificially synthesized peptide
QLENFTLKV

SEQ ID NO: 13 - protein, 9 aa, Artificial Sequence
  An artificially synthesized peptide
TVHLFIPAL

SEQ ID NO: 14 - protein, 9 aa, Artificial Sequence
  An artificially synthesized peptide
VIGKGGKTV

SEQ ID NO: 15 - protein, 9 aa, Artificial Sequence
  An artificially synthesized peptide
IMHKEAQDI

SEQ ID NO: 16 - protein, 9 aa, Artificial Sequence
  An artificially synthesized peptide
KQLSRFAGA

SEQ ID NO: 17 - protein, 9 aa, Artificial Sequence
  An artificially synthesized peptide
GVVESCEQV

SEQ ID NO: 18 - protein, 9 aa, Artificial Sequence
  An artificially synthesized peptide
LQNLSSAEV

SEQ ID NO: 19 - protein, 9 aa, Artificial Sequence
  An artificially synthesized peptide
HLFIPALSV

SEQ ID NO: 20 - protein, 9 aa, Artificial Sequence
  An artificially synthesized peptide
YNPERTITV

SEQ ID NO: 21 - DNA, 4168 bp, Homo sapiens
aagacttaggaagactggtggatgcgtttgggttgtagctaggctttttcttttctttctcttttaaaacacatctagacaaggaaaaaacaagcctcggatctgatttttcactcctcgttcttgtgcttggttcttactgtgtttgtgtattttaaaggcgagaagacgaggggaacaaaaccagctggatccatccatcaccgtgggtggttttaatttttcgttttttctcgttatttttttttaaacaaccactcttcacaatgaacaaactgtatatcggaaacctcagcgagaacgccgccccctcggacctagaaagtatcttcaaggacgccaagatcccggtgtcgggacccttcctggtgaagactggctacgcgttcgtggactgcccggacgagagctgggccctcaaggccatcgaggcgctttcaggtaaaatagaactgcacgggaaacccatagaagttgagcactcggtcccaaaaaggcaaaggattcggaaacttcagatacgaaatatcccgcctcatttacagtgggaggtgctggatagtttactagtccagtatggagtggtggagagctgtgagcaagtgaacactgactcggaaactgcagttgtaaatgtaacctattccagtaaggaccaagctagacaagcactagacaaactgaatggatttcagttagagaatttcaccttgaaagtagcctatatccctgatgaaatggccgcccagcaaaaccccttgcagcagccccgaggtcgccgggggcttgggcagaggggctcctcaaggcaggggtctccaggatccgtatccaagcagaaaccatgtgatttgcctctgcgcctgctggttcccacccaatttgttggagccatcataggaaaagaaggtgccaccattcggaacatcaccaaacagacccagtctaaaatcgatgtccaccgtaaagaaaatgcgggggctgctgagaagtcgattactatcctctctactcctgaaggcacctctgcggcttgtaagtctattctggagattatgcataaggaagctcaagatataaaattcacagaagagatccccttgaagattttagctcataataactttgttggacgtcttattggtaaagaaggaagaaatcttaaaaaaattgagcaagacacagacactaaaatcacgatatctccattgcaggaattgacgctgtataatccagaacgcactattacagttaaaggcaatgttgagacatgtgccaaagctgaggaggagatcatgaagaaaatcagggagtcttatgaaaatgatattgcttctatgaatcttcaagcacatttaattcctggattaaatctgaacgccttgggtctgttcccacccacttcagggatgccacctcccacctcagggcccccttcagccatgactcctccctacccgcagtttgagcaatcagaaacggagactgttcatctgtttatcccagctctatcagtcggtgccatcatcggcaagcagggccagcacatcaagcagctttctcgctttgctggagcttcaattaagattgctccagcggaagcaccagatgctaaagtgaggatggtgattatcactggaccaccagaggctcagttcaaggctcagggaagaatttatggaaaaattaaagaagaaaactttgttagtcctaaagaagaggtgaaacttgaagctcatatcagagtgccatcctttgctgctggcagagttattggaaaaggaggcaaaacggtgaatgaacttcagaatttgtcaagtgcagaagttgttgtccctcgtgaccagacacctgatgagaatgaccaagtggttgtcaaaataactggtcacttctatgcttgccaggttgcccagagaaaaattcaggaaattctgactcaggtaaagcagcaccaacaacagaaggctctgcaaagtggaccacctcagtcaagacggaagtaaaggctcaggaaacagcccaccacagaggcagatgccaaaccaaagacagattgcttaaccaacagatgggcgctgaccccctatccagaatcacatgcacaagtttttacctagccagttgtttctgaggaccaggcaacttttgaactcctgtctctgtgagaatgtatactttatgctctctgaaatgtatgacacccagctttaaaacaaacaaacaaacaaacaaaaaaagggtgggggagggagggaaagagaagagctctgcacttccctttgttgtagtctcacagtataacagatattctaattcttcttaatattcccccataatgccagaaattggcttaatgatgctttcactaaattcatcaaatagattgctcctaaatccaattgttaaaattggatcagaataattatcacaggaacttaaatgttaagccattagcatagaaaaactgttctcagttttatttttacctaacactaacatgagtaacctaagggaagtgctgaatggtgttggcaggggtattaaacgtgcatttttactcaactacctcaggtattcagtaatacaatgaaaagcaaaattgttccttttttttgaaaattttatatactttataatgatagaagtccaaccgttttttaaaaaataaatttaaaatttaacagcaatcagctaacaggcaaattaagatttttacttctggctggtgacagtaaagctggaaaattaatttcagggttttttgaggcttttgacacagttattagttaaatcaaatgttcaaaaatacggagcagtgcctagtatctggagagcagcactaccatttattctttcatttatagttgggaaagtttttgacggtactaacaaagtggtcgcaggagattttggaacggctggtttaaatggcttcaggagacttcagttttttgtttagctacatgattgaatgcataataaatgctttgtgcttctgactatcaatacctaaagaaagtgcatcagtgaagagatgcaagactttcaactgactggcaaaaagcaagctttagcttgtcttataggatgcttagtttgccactacacttcagaccaatgggacagtcatagatggtgtgacagtgtttaaacgcaacaaaaggctacatttccatggggccagcactgtcatgagcctcactaagctattttgaagatttttaagcactgataaattaaaaaaaaaaaattagactccaccttaagtagtaaagtataacaggatttctgtatactgtgcaatcagttctttgaaaaaaaagtcaaaagatagagaatacaagaaaagtttttgggatataatttgaatgactgtgaaaacatatgacctttgataacgaactcatttgctcactccttgacagcaaagcccagtacgtacaattgtgttgggtgtgggtggtctccaaggccacgctgctctctgaattgattttttgagttttgtttgtaagatgatcacagtcatgttacactgatctaaaggacatatatataaccctttaaaaaaaaaatcactgcctcattcttatttcaagatgaatttctatacagactagatgtttttctgaagatcaattagacattttgaaaatgatttaaagtgttttccttaatgttctctgaaaacaagtttcttttgtagttttaaccaaaaaagtgccctttttgtcactggattctcctagcattcatgatttttttttcatacaatgaattaaaattgctaaaatcatggactggctttctggttggatttcaggtaagatgtgtttaaggccagagcttttctcagtatttgatttttttccccaatatttgattttttaaaaatatacacataggtgctgcatttatatctgctggtttaaattctgtcatatttcacttctagccttttagtatggcaaatcatattttacttttacttaagcatttgtaatttggagtatctggtactagctaagaaataattctataattgagttttgtactcaccatatatggatcattcctcatgtataatgtgccccaaatgcagcttcattttccagataccttgacgcagaataaattttttcatcatttaggtgcaaaaaaaaaa

SEQ ID NO: 22 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 23 - DNA, 22 bp, Artificial Sequence
  an artificially synthesized sequence
gtctaccaggcattcgcttcat

SEQ ID NO: 24 - DNA, 24 bp, Artificial Sequence
  an artificially synthesized sequence
tcagctggaccacagccgcagcgt

SEQ ID NO: 25 - DNA, 21 bp, Artificial Sequence
  an artificially synthesized sequence
tcagaaatcctttctcttgac

SEQ ID NO: 26 - DNA, 24 bp, Artificial Sequence
  an artificially synthesized sequence
ctagcctctggaatcctttctctt

(19) United States
(12) Patent Application Publication
(10) Pub. No.: US 2012/0308590 A1
(43) Pub. Date: Dec. 6, 2012

(54) IMP-3 OLIGOPEPTIDES AND VACCINES INCLUDING THE SAME

(75) Inventors: Yasuharu Nishimura, Kumamoto (JP); Michiko Harao, Kumamoto (JP); Yusuke Tomita, Kumamoto (JP); Yusuke Nakamura, Tokyo (JP); Takuya Tsunoda, Kanagawa (JP)

(73) Assignee: OncoTherapy Science, Inc., Kawasaki-shi (JP)

(21) Appl. No.: 13/513,120

(22) PCT Filed: Nov. 30, 2010
(86) PCT No.: PCT/JP2010/006966

§ 371 Date: Aug. 13, 2012

Related U.S. Application Data
(60) Provisional application No. 61/265,657, filed on Dec. 1, 2009.
(60) Provisional application No. 61/371,434, filed on Aug. 6, 2010.

(51) Int. Cl. A61K 39/00; C07H 21/04; A61K 31/7088; A61P 35/00; A61K 35/14; C07K 7/06; C12N 5/078

(52) U.S. Cl. 424/185.1; 530/328; 536/23.1; 435/375

(57) ABSTRACT

Oligopeptides having cytotoxic T cell inducibility and suitable for use in the context of cancer immunotherapy, more particularly cancer vaccines are described herein. Notable examples include oligopeptides having the amino acid sequence of SEQ ID NO: 1, 3, 5 or 6, wherein 1, 2, or several amino acids are optionally substituted, deleted, inserted or added so long as they retain the cytotoxic T cell inducibility of the original oligopeptides. Pharmaceutical formulations or “drugs” related to such oligopeptides suitable for treating or preventing cancers or tumors, as well as the post-operative recurrence thereof, are also described.

### PRIORITY

The present application claims the benefit of U.S. Provisional Application No. 61/265,657, filed on Dec. 1, 2009 and U.S. Provisional Application No. 61/371,434, filed on Aug. 6, 2010, the entire contents of which are incorporated by reference herein.

### TECHNICAL FIELD

The present invention relates to the field of biological science, more specifically to the field of cancer therapy. In particular, the present invention relates to novel oligopeptides that are extremely effective as cancer vaccines, and drugs for treating and preventing tumors.

### BACKGROUND ART

It has been demonstrated that CD8 positive cytotoxic T-lymphocytes (CTLs) recognize epitope peptides derived from the tumor-associated antigens (TAAs) found on the major histocompatibility complex (MHC) class I molecules, and then kill the tumor cells. Since the discovery of the melanoma antigen (MAGE) family as the first example of TAAs, many other TAAs have been discovered, primary through immunological approaches (NPL 1, Boon T, Int J Cancer 1993 May 8, 54(2): 177-80; NPL 2, Boon T & van der Bruggen P, J Exp Med 1996 Mar. 1, 183 (3): 725-9). Some of these TAAs are currently undergoing clinical development as immunotherapeutic targets.

Identification of new TAAs, capable of inducing potent and specific anti-tumor immune responses warrants further development and clinical investigation of peptide vaccination strategies for various types of cancer is ongoing (NPL 3, Harris C C, J Natl Cancer Inst 1996 Oct. 16, 88(20): 1442-55; NPL 4, Butterfield L H et al., Cancer Res 1999 Jul. 1, 59 (13): 3134-42; NPL 5, Vissers J L et al., Cancer Res 1999 Nov. 1, 59 (21): 5554-9; NPL 6, van der Burg S H et al., J Immunol 1996 May 1, 156 (9): 3308-14; NPL 7, Tanaka F et al., Cancer Res 1997 Oct. 15, 57(20): 4465-8; NPL 8, Fujie T et al., Int J Cancer 1999 Jan. 18, 80 (2): 169-72; NPL 9, Kikuchi M et al., Int J Cancer 1999 May 5, 81 (3): 459-66; NPL 10, Oiso M et al., Int J Cancer 1999 May 5, 81 (3): 387-94). To date, there have been several reports of clinical trials using these tumor-associated antigen derived peptides. Unfortunately, a low objective response rate has been observed in these cancer vaccine trials so far (NPL 11, Belli F et al., J Clin Oncol 2002 Oct. 15, 20 (20): 4169-80; NPL 12, Coulie P G et al., Immunol Rev 2002 October, 188: 33-42; NPL 13, Rosenberg S A et al., Nat Med 2004 September, 10(9): 909-15). Therefore, there remains a need for the identification of novel TAAs useful as immunotherapeutic targets.

To that end, through gene expression profiling with a genome-wide cDNA microarray containing 23,040 genes, IMP-3 (insulin-like growth factor II mRNA binding protein 3) has been identified as an up-regulated gene in lung and esophageal cancer (NPL 14, T. Kikuchi et al., Oncogene. 2003 Apr. 10; 22 (14): 2192-205, PTL 1, WO2004/031413, PTL 2, WO2007/013665, PTL 3, WO2007/013671). Expression of IMP-3 has been observed to be specifically up-regulated in the tumor cells of more than 90% of the cancer patients but not expressed in other normal vital organs, except for testis and placenta. Furthermore, down-regulation of IMP-3 expression with RNA interference method has been shown to suppress cell growth in IMP-3 expressing cancer cell lines. A previous application, WO2006/090810, describes peptides derived from IMP-3 (also described as KOC1) having specific CTL inducing activity against tumor cells exogenously expressing KOC1 (IMP-3) and HLA-A24. Although these peptides may be suitable for patients of the HLA-A24 type, there remains a need for CTL inducing peptides for other HLA type patients.

### CITATION LIST

### Patent Literature

[PTL 1] WO2004/031413
[PTL 2] WO2007/013665
[PTL 3] WO2007/013671
[PTL 4] WO2006/090810

### Non Patent Literature

[NPL 1] Boon T, Int J Cancer 1993 May 8, 54(2): 177-80
[NPL 2] Boon T & van der Bruggen P, J Exp Med 1996 Mar. 1, 183 (3): 725-9
[NPL 3] Harris C C, J Natl Cancer Inst 1996 Oct. 16, 88(20): 1442-55
[NPL 4] Butterfield L H et al., Cancer Res 1999 Jul. 1, 59(13): 3134-42
[NPL 5] Vissers J L et al., Cancer Res 1999 Nov. 1, 59(21): 5554-9
[NPL 6] van der Burg S H et al., J Immunol 1996 May 1, 156(9): 3308-14
[NPL 7] Tanaka F et al., Cancer Res 1997 Oct. 15, 57(20): 4465-8
[NPL 8] Fujie T et al., Int J Cancer 1999 Jan. 18, 80(2): 169-72
[NPL 9] Kikuchi M et al., Int J Cancer 1999 May 5, 81 (3): 459-66
[NPL 10] Oiso M et al., Int J Cancer 1999 May 5, 81 (3): 387-94
[NPL 11] Belli F et al., J Clin Oncol 2002 Oct. 15, 20(20): 4169-80
[NPL 12] Coulie P G et al., Immunol Rev 2002 October, 188: 33-42
[NPL 13] Rosenberg S A et al., Nat Med 2004 September, 10(9): 909-15
[NPL 14] T. Kikuchi et al., Oncogene. 2003 Apr. 10; 22(14): 2192-205

### SUMMARY OF INVENTION

The present invention is based in part on the discovery of novel peptides that may serve as targets of immunotherapy. Because TAAs are generally perceived by the immune system as “self” and therefore often have no innate immunogenicity, the discovery of appropriate targets is of extreme importance. Recognizing that IMP-3 has been identified as up-regulated in cancers such as lung cancer and esophageal cancer, the present invention targets the IMP-3 protein (SEQ ID NO: 22) encoded by the gene of GenBank Accession No. NM_{—}006547.2 (SEQ ID NO: 21) for further analysis. In particular, IMP-3 gene products containing epitope peptides that elicit surprisingly strong CTL responses specific to the corresponding molecules were selected for study. In the context of the present invention, peripheral blood mononuclear cells (PBMCs) obtained from a healthy donor were stimulated using the peptides of the present invention. CTLs that specifically recognize HLA-A2 (A*0201) positive target cells pulsed with the respective peptides were established, and HLA-A2 (A*0201) restricted epitope peptides that can induce potent and specific immune responses against IMP-3 expressed on the surface of tumor cells were identified. Take together, these results demonstrate that IMP-3 is strongly immunogenic and the epitopes thereof are effective targets for tumor immunotherapy.

Accordingly, it is an object of the present invention to provide oligopeptides having CTL inducibility as well as an amino acid sequence selected from among SEQ ID NOs: 1, 3, 5 and 6. In addition, the present invention contemplates modified peptides, having an amino acid sequence of SEQ ID NOs: 1, 3, 5 or 6, wherein one, two or several amino acids are mutated or altered by at least one of mutation selected from the group consisting of substitution, deletion, insertion and addition, so long as the resulting modified oligopeptides retain the CTL inducibility of the original peptides.

When administered to a subject, the present oligopeptides are presented on the surface of antigen-expressing cells so as to induce CTLs targeting the respective peptides. Therefore, it is an object of the present invention to provide antigen-presenting cells and exosomes that present any of the present peptides, as well as methods for inducing antigen-presenting cells associated therewith.

An anti-tumor immune response is induced by the administration of the present IMP-3 oligopeptides or polynucleotides encoding the oligopeptides, as well as exosomes and antigen-presenting cells which present such IMP-3 oligopeptides. Therefore, it is yet another object of the present invention to provide pharmaceutical agents or pharmaceutical compositions containing the oligopeptides or polynucleotides encoding them, or the associated exosomes and antigen-presenting cells, as their active ingredients. The pharmaceutical agents or pharmaceutical compositions of the present invention find particular use as vaccines.

It is a further object of the present invention to provide methods for at least one of purpose selected from group consisting of treatment, prophylaxis of (i.e., prevention) cancers (tumors), and prevention of the postoperative recurrence thereof, as well as methods for inducing CTLs, methods for inducing anti-tumor immunity, such methods including the step of administering the IMP-3 oligopeptides, polynucleotides encoding IMP-3 oligopeptides, exosomes or the antigen-presenting cells presenting IMP-3 polypeptides or the pharmaceutical agents or compositions of the present invention, to a subject in need thereof. In addition, the CTLs of the present invention also find use as vaccines against cancer. Examples of target cancers include, but are not limited to lung cancer and esophageal cancer.

More specifically, the present invention provides followings:

[1] An isolated oligopeptide comprising an amino acid sequence selected from the group consisting of SEQ ID NOs: 1, 3, 5 and 6,

[2] An isolated oligopeptide comprising an amino acid sequence selected from the group consisting of SEQ ID NOs: 1, 3, 5 and 6, wherein 1, 2, or several amino acid(s) are substituted, deleted, inserted and/or added, further wherein the oligopeptide has cytotoxic T lymphocyte (CTL) inducibility,

[3] The oligopeptide of [2], wherein the oligopeptide has one or both of the following characteristics:
(a) the second amino acid from the N-terminus is leucine or methionine, and
(b) the C-terminal amino acid is valine or leucine,

[4] An isolated polynucleotide encoding the oligopeptide of any one of [1] to [3],

[5] A method for inducing an antigen-presenting cell having CTL inducibility by using an oligopeptide as set forth in any one of [1] to [3],

[6] The method of [5], wherein the method comprises a step selected from the group consisting of:

(a) contacting an antigen-presenting cell with the oligopeptide of any one of [1] to [3], and

(b) introducing a polynucleotide encoding the oligopeptide of any one of [1] to [3] into an antigen-presenting cell,

[7] The method of [5] or [6], wherein the antigen presenting cell expresses at least one HLA-A2 antigen on its surface,

[8] A method for inducing CTL by using the oligopeptide as set forth in any one of [1] to [3],

[9] The method of [8], wherein the method comprises a step selected from the group consisting of:
(a) contacting a CD8-positive T cell with an antigen-presenting cell and/or an exosome that presents a complex of the oligopeptide of any one of [1] to [3] and an HLA antigen on its surface, and
(b) introducing a polynucleotide encoding a polypeptide that is capable of forming a T cell receptor (TCR) subunit binding to a complex of the oligopeptide of any one of [1] to [3] and an HLA antigen on an antigen-presenting cell surface, into a CD8-positive T cell,

[10] The method of [9], wherein the HLA antigen is HLA-A2,

[11] An isolated CTL that targets the oligopeptide of any one of [1] to [3],

[12] The CTL of [11], wherein said CTL is capable of binding to a complex of the oligopeptide of any one of [1] to [3] and an HLA antigen on a cell surface,

[13] The CTL of [12], wherein said HLA antigen is HLA-A2,

[14] An isolated CTL that is induced by using the oligopeptide of any one of [1] to [3],

[15] The CTL of [14], wherein said CTL is induced by the method of any one of [8] to [10],

[16] An isolated antigen-presenting cell that presents on its surface a complex of an HLA antigen and the oligopeptide of any one of [1] to [3],

[17] The antigen-presenting cell of [16], wherein the HLA antigen is HLA-A2,

[18] The antigen-presenting cell of [16] or [17], wherein said antigen-presenting cell is induced by any one of the method of [5] to [7],

[19] A method of inducing an immune response against a cancer in a subject, the method comprising the step of administering to the subject a vaccine comprising at least one active ingredient selected from the group consisting of:
(a) one or more oligopeptide(s) of any one of [1] to [3], or an immunologically active fragment thereof;
(b) one or more polynucleotide(s) encoding the oligopeptide of any one of [1] to [3], or an immunologically active fragment thereof;
(c) one or more isolated CTL(s) of any one of [11] to [15]; and

(d) one or more isolated antigen-presenting cell(s) of any one of [16] to [18],

[20] The method of [19], wherein said subject is HLA-A2 positive,

[21] A pharmaceutical agent for the treatment and/or prophylaxis of cancer, and/or the prevention of a postoperative recurrence thereof, wherein the agent comprises a pharmaceutically acceptable carrier and at least one active ingredient(s) selected from the group consisting of:
(a) one or more oligopeptide(s) of any one of [1] to [3], or an immunologically active fragment thereof;
(b) one or more polynucleotide(s) encoding the oligopeptide of any one of [1] to [3], or an immunologically active fragment thereof;
(c) one or more antigen-presenting cell(s) presenting a complex of the oligopeptide of any one of [1] to [3] and an HLA antigen; and
(d) one or more CTL(s) that is capable of binding to a complex of the oligopeptide of any one of [1] to [3] and an HLA antigen on a cell surface,

[22] A pharmaceutical agent for inducing CTLs, wherein the agent comprises a pharmaceutically acceptable carrier and at least one active ingredient(s) selected from the group consisting of:
(a) one or more oligopeptide(s) of any one of [1] to [3], or an immunologically active fragment thereof;
(b) one or more polynucleotide(s) encoding the oligopeptide of any one of [1] to [3], or an immunologically active fragment thereof;
(c) one or more antigen-presenting cell(s) presenting a complex of the oligopeptide of any one of [1] to [3] and an HLA antigen,

[23] The pharmaceutical agent of [21] or [22], wherein the pharmaceutical agent is formulated for the administration to a subject who is HLA-A2 positive,

[24] The pharmaceutical agent of any one of [21] to [23], which is a vaccine,

[25] Use of an active ingredient selected from the group consisting of:
(a) one or more oligopeptide(s) of any one of [1] to [3];
(b) one or more polynucleotide(s) encoding the oligopeptide of any one of [1] to [3] in an expressible form;
(c) one or more antigen-presenting cell(s) presenting a complex of the oligopeptide of any one of [1] to [3] and HLA antigen on its surface; and
(d) one or more CTL(s) that is capable of binding to a complex of the oligopeptide of any one of [1] to [3] and an HLA antigen on a cell surface, in manufacturing a pharmaceutical composition or agent for treating cancer, and

[26] The use of [25], wherein the pharmaceutical composition or agent is formulated for the administration to a subject who is HLA-A2 positive,

[27] An isolated oligopeptide comprising an amino acid sequence selected from the group consisting of SEQ ID NOs: 1, 3, 5 and 6, for use in the treatment and/or prophylaxis of cancer, and/or the prevention of a postoperative recurrence thereof in a subject who is HLA-A2 positive,

[28] An isolated oligopeptide comprising an amino acid sequence selected from the group consisting of SEQ ID NOs: 1, 3, 5 and 6, wherein 1, 2, or several amino acid(s) are substituted, deleted, inserted and/or added, further wherein the oligopeptide has cytotoxic T lymphocyte (CTL) inducibility, for use in the treatment and/or prophylaxis of cancer, and/or the prevention of a postoperative recurrence thereof in a subject who is HLA-A2 positive, and

[29] The oligopeptide of [28], wherein the oligopeptide has one or both of the following characteristics:
(a) the second amino acid from the N-terminus is leucine or methionine, and
(b) the C-terminal amino acid is valine or leucine.

In addition to the above, other objects and features of the invention will become more fully apparent when the following detailed description is read in conjunction with the accompanying figures and examples. However, it is to be understood that both the foregoing summary of the invention and the following detailed description are of exemplified embodiments, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments which follows.

FIG. 1 depicts the results of an IFN-gamma ELISPOT assay on CTLs that were induced in HLA-A2 transgenic mice. CTLs stimulated with peptides (SEQ ID NOs: 3, 5 and 6) showed potent IFN-gamma productive responses as compared with the controls (upper panel). Error bars represent standard deviation (SD). Statistically significant differences are indicated with asterisks (* P<0.05). Exemplary photographs of ELISPOT counts of triplicate wells are also shown (lower panel). The CTLs showed 203 to 226 spots/well in response to BM-DC pulsed with the peptide of SEQ ID NO: 6 (panels of leftside), whereas they showed 74 to 105 spots/well in the presence of BM-DC without peptide loading (panels of rightside).

FIG. 2 is composed of a series of bar graphs depicting the results of an IFN-gamma ELISPOT assay on human CTLs of healthy donor 1. Human CTLs stimulated with peptides of SEQ ID NOs: 1, 3, 5 and 6 showed potent IFN-gamma productive responses against T2 cells pulsed with cognate peptides as compared with that pulsed with irrelevant HIV peptide (P<0.05). Error bars represent SD.

FIG. 3 is composed of a series of distribution (A) and line (B) graphs depicting the induction of IMP-3-specific human CTLs from CD8⁺ T cells of HLA-A2-positive lung cancer patients and healthy donors. Part (A) presents the results of FACS (fluorescence-activated cell sorter) analysis to detect the expression of CD107a on the cell surface of human CTLs of healthy donor 1 or lung cancer patient 1 after stimulation with peptide of SEQ ID NOs: 1, 3 or 6. CTLs stimulated with the peptide were stained with FITC (fluorescein isothiocyanate)-conjugated anti-CD107a antibody (upper panel) or FITC-conjugated anti-mouse IgG1 as control (middle panel). As negative control of stimulation, CTLs were stimulated with HIV peptide and stained with FITC-conjugated anti-CD107a antibody (lower panel). Expression of CD107a was detected on CTLs when they were stimulated with the peptide SEQ ID NO: 1, 3 or 6 as compared with control. Part (B) depicts the cytotoxicity of IMP-3-specific CTLs against T2 cells pulsed with the cognate IMP-3-derived peptides. Cytotoxicity of CTLs against T2 cells pulsed with the peptide of SEQ ID NO: 1 (open triangle; left and middle panels) or the peptide of SEQ ID NO: 6 (open triangle; right panel) and T2 cells pulsed with irrelevant HIV-A2 peptides (closed triangle) in ⁵¹Cr-release assay. Each value represents the percentage of specific lysis calculated based on the mean values of a triplicate assay.

FIG. 4 is composed of a series of bar (A) and line (B) graphs depicting induction of IMP-3-specific CTLs from PBMCs of three lung cancer patients. Part (A) depicts CTLs induced from PBMCs of patient 14 by stimulation with peptide of SEQ ID NO: 5 and patient 103 with peptide of SEQ ID NO: 6 showed significant IFN-gamma production against T2 cells pulsed with cognate peptides as compared with that pulsed with irrelevant HIV peptide. Statistically significant differences are indicated with asterisks (* P<0.05). Error bars represent SD. Part (B) depicts CTLs induced from PBMCs of lung cancer patient 4 with peptide of SEQ ID NO: 3 and patient 3 with peptide of SEQ ID NO: 5 showed cytotoxic activity against T2 cells pulsed with cognate peptides as compared with those pulsed with irrelevant HIV peptide.

FIG. 5 is composed of a series of line graphs depicting the results of ⁵¹Cr release assay using CTLs and tumor cell lines endogenously expressing IMP-3. Part (A) presents the cytotoxic activities of CTLs induced from PBMCs of healthy donor 2 by stimulation with peptides of SEQ ID NOs: 1, 3, 5 and 6 are shown. These CTLs showed cytotoxic activity against PANC-1 (IMP-3⁺, HLA-A2⁺), but showed no cytotoxic activity against MCF7 (IMP-3⁻, HLA-A2⁺) and A549 (IMP-3⁺, HLA-A2⁻). Part (B) presents the cytotoxic activities of CTLs induced from PBMCs of lung cancer patient 14 by stimulation with peptides of SEQ ID NOs: 3 and 5, and patient 4 with the peptide of SEQ ID NO: 6 were detected by ⁵¹Cr release assay. These CTLs showed cytotoxic activity against PANC-1 (IMP-3⁺, HLA-A2⁺), but showed no cytotoxic activity against MCF7 (IMP-3⁻, HLA-A2⁺) and A549 (IMP-3⁺, HLA-A2⁻). Part (C) presents the cytotoxic activities of IMP-3-specific CTLs against MCF7/IMP3 (open circle; MCF7 cells transfected with IMP-3 gene) or MCF7 (closed circle) analyzed by ⁵¹Cr-release assay.

FIG. 5D Part (D) presents the cytotoxic activities of IMP-3-specific CTLs against SW620 (open triangle), SKHep1 (open lozenge), MCF7 (closed circle) or A549 (closed lozenge) analyzed by ⁵¹Cr-release assay. The CTL lines generated from the healthy donors by stimulation with either the peptide of SEQ ID NO: 1 or the peptide of SEQ ID NO: 6 exhibited cytotoxic activity against SW620, SKHep1 but not against A549 (HLA-A2−, IMP-3+) or MCF7 cells (HLA-A2+, IMP-3−).

FIG. 6 is composed of a series of bar graphs (A, B, D) and line graphs (C) depicting the inhibition of CTL responses by anti-HLA class I mAb (W6/32, IgG2a) or anti-HLA-A2 mAb. CTL activities induced from PBMCs of lung cancer patient 14 by stimulation with peptides SEQ ID NOs: 1, 3, 5 and 6 were detected by IFN-gamma ELISPOT assay (A). The IFN-gamma production mediated by the CTLs was markedly inhibited by W6/32, whereas no inhibition of IFN-gamma production was detected by treatment with anti-HLA-DR mAb (H-DR-1, IgG2a). Error bars represent SD. Statistically significant differences are indicated with asterisks (* P<0.05). IFN-gamma production (B) and cytotoxicity (C and D) mediated by CTLs are indicated. Open circle, PANC1; Closed circle, PANC1+W6/32; Square, PANC1+control mAb. Bars indicate the IFN-gamma production (B) or cytotoxicity (D) when the generated CTL lines were co-cultured with PANC1 (open bars), PANC1+control mAb (open bars) or PANC1+blocking mAb (closed bars). Representative data from two independent experiments with similar results is shown. Statistically significant differences in (B) are indicated with asterisks.

FIG. 6C-D is the continuation of FIG. 6A-B.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

Unless otherwise defined, all technical and scientific terms used herein have the same meaning as commonly understood by one of ordinary skill in the art to which the present invention belongs. In case of conflict, the present specification, including definitions, will control. In addition, the materials, methods, and examples are illustrative only and not intended to be limiting.

### I. Definitions

The words “a”, “an”, and “the” as used herein mean “at least one” unless otherwise specifically indicated.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “oligopeptide” sometimes used in the present specification is used to refer to peptide which are 20 residues or fewer, typically 15 residues or fewer in length and is typically composed of between about 8 and about 11 residues, often 9 or 10 residues. Through the present specification, the term “peptide” is used for the same meaning as the term “oligopeptide” unless otherwise specifically indicated.

The term “amino acid” as used herein refers to naturally occurring and synthetic amino acids, as well as amino acid analogs and amino acid mimetics that have similarly function to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “nucleotides” and “nucleic acids” are used interchangeably herein unless otherwise specifically indicated and are similarly to the amino acids referred to by their commonly accepted single-letter codes.

The terms “agent” and “composition” are used interchangeably herein to refer to a product including the specified ingredients in the specified amounts, as well as any product that results, directly or indirectly, from combination of the specified ingredients in the specified amounts. Such terms in relation to the modifier “pharmaceutical” are intended to encompass a product including the active ingredient(s), and any inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, in the context of the present invention, the terms “pharmaceutical agent” and “pharmaceutical composition” are used interchangeably to refer to any agent, substance or composition made by admixing a product of the present invention and a pharmaceutically or physiologically acceptable carrier. The phrase “pharmaceutically acceptable carrier” or “physiologically acceptable carrier”, as used herein, means a pharmaceutically or physiologically acceptable material, composition, substance or vehicle, including but not limited to, a liquid or solid filler, diluent, excipient, solvent or encapsulating material, involved in carrying or transporting the subject scaffolded polypharmacophores from one organ, or portion of the body, to another organ, or portion of the body.

The pharmaceutical agents or compositions of the present invention find particular use as vaccines. In the context of the present invention, the phrase “vaccine” (also referred to as an “immunogenic composition”) refers to a substance that has the function to induce anti-tumor immunity upon inoculation into animals.

The term “active ingredient” herein refers to a substance in an agent or composition that is biologically or physiologically active. Particularly, in a pharmaceutical agent or composition, “active ingredient” refers to a substance that shows an objective pharmacological effect. For example, in case of pharmaceutical agents or compositions for use in the treatment or prevention of cancer, active ingredients in the agents or compositions may lead to at least one biological or physiologically action on cancer cells and/or tissues directly or indirectly. Preferably, such action may include reducing or inhibiting cancer cell growth, damaging or killing cancer cells and/or tissues, and so on. Typically, indirect effect of active ingredients is inductions of CTLs recognizing or killing cancer cells. Before formulated, “active ingredient” is also referred to as “bulk”, “drug substance” or “technical product”.

Unless otherwise defined, the term “cancer” refers to the cancers over-expressing the IMP-3 gene, examples of which include, but are not limited to, lung cancer and esophageal cancer.

Unless otherwise defined, the term “cytotoxic T lymphocyte”, “cytotoxic T cell” and “CTL” are used interchangeably herein and, unless otherwise specifically indicated, refer to a sub-group of T lymphocytes that are capable of recognizing non-self cells (e.g., tumor cells, virus-infected cells) and inducing the death of such cells. Unless otherwise defined, the term “kit” as used herein, is used in reference to a combination of reagents and other materials. It is contemplated herein that the kit may include microarray, chip, marker, and so on. It is not intended that the term “kit” be limited to a particular combination of reagents and/or materials.

As used herein, in the context of a subject or patient, the phrase “HLA-A2 positive” refers to that the subject or patient homozygously or heterozygously possess HLA-A2 antigen gene, and HLA-A2 antigen is expressed in cells of the subject or patient as an HLA antigen.

To the extent that the methods and compositions of the present invention find utility in the context of the “treatment” of cancer, a treatment is deemed “efficacious” if it leads to clinical benefit such as, reduction in expression of IMP-3 gene expression, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods and compositions of the present invention find utility in the context of the “prevention” and “prophylaxis” of cancer, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

In the context of the present invention, the treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effective treatment and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or achieving a stable disease state.

In the context of the present invention, the term “antibody” refers to immunoglobulins and fragments thereof that are specifically reactive to a designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity. An “antibody” indicates all classes (e.g., IgA, IgD, IgE, IgG and IgM).

### II. Peptides

To demonstrate that peptides derived from IMP-3 function as an antigen recognized by cytotoxic T lymphocytes (CTLs), peptides derived from IMP-3 (SEQ ID NO: 22) were analyzed to determine whether they were antigen epitopes restricted by HLA-A2 (ex. A*0201 and A*0206) which are commonly encountered HLA alleles (Date Y et al., Tissue Antigens 47: 93-101, 1996; Kondo A et al., J Immunol 155: 4307-12, 1995; Kubo R T et al., J Immunol 152: 3913-24, 1994). Candidates of HLA-A2 binding peptides derived from IMP-3 were identified based on their binding affinities to HLA-A2. After in vitro stimulation of T-cells by dendritic cells (DCs) loaded with these peptides, CTLs were successfully established using each of the peptides, particularly the peptides of SEQ ID NOs: 1, 3, 5 and 6.

These established CTLs show potent specific CTL activity against target cells pulsed with respective peptides and also cells expressing HLA-A*0201 and IMP-3. These results herein demonstrate that IMP-3 is an antigen recognized by CTL and that the peptides may be epitope peptides of IMP-3 restricted by HLA-A2 (ex. A*0201 and A*0206).

Since the IMP-3 gene is over expressed in most cancer tissues, such as lung cancer and esophageal cancer, it is a good target for immunotherapy. Thus, the present invention provides oligopeptides such as nonapeptides (peptides composed of nine amino acid residues) and decapeptides (peptides composed of ten amino acid residues) corresponding to CTL-recognized epitopes of IMP-3. Particularly preferred examples of oligopeptides of the present invention include peptides having an amino acid sequence selected from among SEQ ID NOs: 1, 3, 5 and 6.

Generally, software programs presently available on the Internet, such as those described in Parker K C et al., J Immunol 1994 Jan. 1, 152 (1): 163-75, can be used to calculate the binding affinities between various peptides and HLA antigens in silico. Binding affinity with HLA antigens can be measured as described, for example, in the reference of Parker K C et al., J Immunol 1994 Jan. 1, 152 (1): 163-75; and Kuzushima K et al., Blood 2001, 98(6): 1872-81. Methods for determining binding affinity are described, for example, in the Journal of Immunological Methods, 1995, 185: 181-190 and Protein Science, 2000, 9: 1838-1846. Thus, the present invention encompasses peptides of IMP-3 that bind with HLA antigens identified using such known programs.

The oligopeptides of the present invention can be flanked with additional amino acid residues so long as the resulting peptide retains its CTL inducibility. Such peptides having CTL inducibility are typically less than about 40 amino acids, often less than about 20 amino acids, usually less than about 15 amino acids. The particular amino acid sequences flanking the oligopeptides of the present invention (e.g., oligopeptides composed of the amino acid sequence selected from among SEQ ID NOs: 1, 3, 5 and 6) is not limited and can be composed of any kind of amino acids so long as it does not impair the CTL inducibility of the original peptide. Thus, the present invention also provides peptides having CTL inducibility and the amino acid sequence selected from among SEQ ID NOs: 1, 3, 5 and 6.

In general, the modification of one, two, or several amino acids in a protein will not influence the function of the protein, and in some cases will even enhance the desired function of the original protein. In fact, modified peptides (i.e., peptides composed of an amino acid sequence in which one, two or several amino acid residues have been modified (i.e., substituted, deleted, added and/or inserted) as compared to an original reference sequence) have been known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the oligopeptides of the present invention may have both CTL inducibility and an amino acid sequence selected from among SEQ ID NOs: 1, 3, 5 and 6 wherein one, two or several amino acids are added, inserted, deleted, and/or substituted.

Those of skill in the art recognize that individual additions or substitutions to an amino acid sequence which alters a single amino acid or a small percentage of amino acids tend to result in the conservation of the properties of the original amino acid sidechain. As such, they are conventionally referred to as “conservative substitutions” or “conservative modifications”, wherein the alteration of a protein results in a modified protein having properties and functions analogous to the original protein. Conservative substitution tables providing functionally similar amino acids are well known in the art. Examples amino acid side chain characteristics that are desirable to conserve include, for example, hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing sidechain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins 1984).

Such conservatively modified peptides are also considered to be peptides of the present invention. However, peptides of the present invention are not restricted thereto and can include non-conservative modifications, so long as the modified peptide retains the CTL inducibility of the original peptide. Furthermore, modified peptides should not exclude CTL inducible peptides of polymorphic variants, interspecies homologues, and alleles of IMP-3.

To retain the requisite CTL inducibility one can modify (insert, delete, add and/or substitute) a small number (for example, 1, 2 or several) or a small percentage of amino acids. Herein, the term “several” means 5 or fewer amino acids, for example, 4 or 3 or fewer. The percentage of amino acids to be modified is preferably 20% or less, more preferably 15% or less, even more preferably 10% or less or 1 to 5%.

When used in the context of immunotherapy, peptides of the present invention should be presented on the surface of a cell or exosome, preferably as a complex with an HLA antigen. Therefore, it is preferable to select peptides that not only induce CTLs but also possess high binding affinity to the HLA antigen. To that end, the peptides can be modified by substitution, insertion, deletion, and/or addition of the amino acid residues to yield a modified peptide having improved binding affinity. In addition to peptides that are naturally displayed, since the regularity of the sequences of peptides displayed by binding to HLA antigens is already known (J Immunol 1994, 152: 3913; Immunogenetics 1995, 41: 178; J Immunol 1994, 155: 4307), modifications based on such regularity can be introduced into the immunogenic peptides of the invention.

For example, it may be desirable to substitute the second amino acid from the N-terminus substituted with leucine or methionine, and/or the amino acid at C-terminus with valine or leucine in order to increase the HLA-A24 binding affinity. Thus, peptides having the amino acid sequences of SEQ ID NOs: 1, 3, 5 and 6 wherein the second amino acid from the N-terminus of the amino acid sequence of the SEQ ID NOs is substituted with leucine or methionine and/or wherein the C-terminus of the amino acid sequence of the SEQ ID NOs is substituted with valine or leucine, are encompassed by the present invention.

Substitutions can be introduced not only at the terminal amino acids but also at the position of potential TCR recognition of peptides. Several studies have demonstrated that amino acid substitutions in a peptide can be equal to or better than the original, for example CAP1, p53₍₂₆₄₋₂₇₂₎, Her-2/neu₍₃₆₉₋₃₇₇₎ or gp 100₍₂₀₉₋₂₁₇₎ (Zaremba et al. Cancer Res. 57, 4570-4577, 1997, T. K. Hoffmann et al. J. Immunol. (2002) February 1; 168(3):1338-47., S. O. Dionne et al. Cancer Immunol immunother. (2003) 52: 199-206 and S. O. Dionne et al. Cancer Immunology, Immunotherapy (2004) 53, 307-314).

The present invention also contemplates the addition of amino acids to the sequences disclosed herein. For example, one, two or several amino acids can also be added to the N and/or C-terminus of the described peptides. Such modified peptides having high HLA antigen binding affinity and retain CTL inducibility are also included in the present invention.

However, when the peptide sequence is identical to a portion of the amino acid sequence of an endogenous or exogenous protein having a different function, side effects such as autoimmune disorders and/or allergic symptoms against specific substances may be induced. Therefore, it is preferable to first perform homology searches using available databases to avoid situations in which the sequence of the peptide matches the amino acid sequence of another protein. When it becomes clear from the homology searches that no peptide exists with as few as 1 or 2 amino acid differences as compared to the objective peptide, the objective peptide can be modified in order to increase its binding affinity with HLA antigens, and/or increase its CTL inducibility without any danger of such side effects.

Although peptides having high binding affinity to the HLA antigens as described above are expected to be highly effective, the candidate peptides, which are selected according to the presence of high binding affinity as an indicator, are further examined for the presence of CTL inducibility. Herein, the phrase “CTL inducibility” indicates the ability of the peptide to induce cytotoxic lymphocytes (CTLs) when presented on antigen-presenting cells. Further, “CTL inducibility” includes the ability of the peptide to induce CTL activation, CTL proliferation, promote CTL lysis of target cells, and to increase CTL IFN-gamma production.

Confirmation of CTL inducibility is accomplished by inducing antigen-presenting cells carrying human MHC antigens (for example, B-lymphocytes, macrophages, and dendritic cells (DCs)), or more specifically DCs derived from human peripheral blood mononuclear leukocytes, and after stimulation with the peptides, mixing with CD8-positive cells, and then measuring the IFN-gamma produced and released by CTLs against the target cells. As the reaction system, transgenic animals that have been produced to express a human HLA antigen (for example, those described in BenMohamed L, Krishnan R, Longmate J, Auge C, Low L, Primus J, Diamond D J, Hum Immunol 2000 August, 61 (8): 764-79, Related Articles, Books, Linkout Induction of CTL response by a minimal epitope vaccine in HLA A*0201/DR1 transgenic mice: dependence on HLA class II restricted T(H) response) can be used. For example, the target cells can be radio-labeled with ⁵¹Cr and such, and cytotoxic activity can be calculated from radioactivity released from the target cells. Alternatively, CTL inducibility can be assessed by measuring IFN-gamma produced and released by CTLs in the presence of antigen-presenting cells (APCs) that carry immobilized peptides, and visualizing the inhibition zone on the media using anti-IFN-gamma monoclonal antibodies.

As a result of examining the CTL inducibility of the peptides as described above, it was discovered that those peptides having high binding affinity to an HLA antigen did not necessarily have high CTL inducibility. However, of those peptides identified and assessed, oligopeptides selected from peptides having an amino acid sequences indicated by SEQ ID NOs: 1, 3, 5 and 6, were found to exhibit particularly high CTL inducibility as well as high binding affinity to an HLA antigen. Thus, these peptides are exemplified as preferred embodiments of the present invention.

In addition to the above-described modifications, the peptides of the present invention can also be linked to other substances, so long as the resulting linked peptide retains the requisite CTL inducibility of the original peptide. Examples of suitable substances include, but are not limited to: peptides, lipids, sugar and sugar chains, acetyl groups, natural and synthetic polymers, etc. The peptides can contain modifications such as glycosylation, side chain oxidation, or phosphorylation, etc. provided the modifications do not destroy the biological activity of the original peptide. These kinds of modifications can be performed to confer additional functions (e.g., targeting function, and delivery function) or to stabilize the polypeptide.

For example, to increase the in vivo stability of a polypeptide, it is known in the art to introduce D-amino acids, amino acid mimetics or unnatural amino acids; this concept can also be adapted to the present polypeptides. The stability of a polypeptide can be assayed in a number of ways. For instance, peptidases and various biological media, such as human plasma and serum, can be used to test stability (see, e.g., Verhoef et al., Eur J Drug Metab Pharmacokin 1986, 11: 291-302).

Further, the peptides of the present invention may be linked to other peptides via spacers or linkers. Examples of other peptides include, but are not limited to, CTL inducible peptides derived from other TAAs. Alternatively, two or more peptides of the present invention may be linked via spacers or linkers. The peptides linked via spacers or linkers may be the same or different to each other. The kind of spacers and linkers is not specifically limited, and include those composed of peptides, more preferably those composed of peptides having one or more cleavage sites which are capable of being cleaved by enzymes such as peptidases, proteases and proteasomes. Examples of linkers or spacers include, but are not limited to: AAY (P. M. Daftarian et al., J Trans Med 2007, 5:26), AAA, NKRK (R. P. M. Sutmuller et al., J. Immunol. 2000, 165: 7308-7315) or, one to several lysine residues (S. Ota et al., Can Res. 62, 1471-1476, K. S. Kawamura et al., J. Immunol. 2002, 168: 5709-5715). The present invention contemplates peptides linked to other peptides via spacers or linkers.

When the peptides of the present intention include a cystein residue, the peptides tend to form dimers via a disulfide bond between SH groups of the cyctein residues. Therefore, dimers of the peptide of the present invention are also included in the peptides of the present invention.

Herein, the peptides of the present invention can also be described as “IMP-3 peptide(s)”, “IMP-3 polypeptide(s)” or “IMP-3 oligopeptide”.

### III. Preparation of IMP-3 Peptides

The peptides of the present invention can be prepared using well known techniques. For example, the peptides can be prepared synthetically, using recombinant DNA technology or chemical synthesis. The peptides of the present invention can be synthesized individually or as longer polypeptides composed of two or more peptides. The peptides can then be isolated i.e., purified or isolated so as to be substantially free of other naturally occurring host cell proteins and fragments thereof, or any other chemical substances.

The peptides of the present invention may also contain modifications, such as glycosylation, side chain oxidation, or phosphorylation provided such modifications do not destroy the biological activity of the original peptide. Other illustrative modifications include incorporation of D-amino acids or other amino acid mimetics that may be used, for example, to increase the serum half life of the peptides.

A peptide of the present invention can be obtained through chemical synthesis based on the selected amino acid sequence. Examples of conventional peptide synthesis methods that can be adapted to the synthesis include, but are not limited to:

### (i) Peptide Synthesis, Interscience, New York, 1966;

### (ii) The Proteins, Vol. 2, Academic Press, New York, 1976;

(iii) Peptide Synthesis (in Japanese), Maruzen Co., 1975;

### (iv) Basics and Experiment of Peptide Synthesis (in Japanese), Maruzen Co., 1985;

(v) Development of Pharmaceuticals (second volume) (in Japanese), Vol. 14 (peptide synthesis), Hirokawa, 1991;

### (vi) WO99/67288; and

(vii) Barany G. & Merrifield R. B., Peptides Vol. 2, “Solid Phase Peptide Synthesis”, Academic Press, New York, 1980, 100-118.

Alternatively, the present peptides can be obtained adapting any known genetic engineering methods for producing peptides (e.g., Morrison J, J Bacteriology 1977, 132: 349-51; Clark-Curtiss & Curtiss, Methods in Enzymology (eds. Wu et al.) 1983, 101: 347-62). For example, first, a suitable vector harboring a polynucleotide encoding the objective peptide in an expressible form (e.g., downstream of a regulatory sequence corresponding to a promoter sequence) is prepared and transformed into a suitable host cell. The host cell is then cultured to produce the peptide of interest. The peptide can also be produced in vitro adapting an in vitro translation system.

### IV. Polynucleotides

The present invention also provides a polynucleotide which encodes any of the aforementioned peptides of the present invention. These include polynucleotides derived from the natural occurring IMP-3 gene (GenBank Accession No. NM_{—}006547.2 (SEQ ID NO: 21)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a peptide also describes every possible silent variation of the nucleic acid. One of ordinary skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence.

The polynucleotide of the present invention can be composed of DNA, RNA, and derivatives thereof. As is well known in the art, a DNA is suitably composed of bases such as the naturally occurring bases A, T, C, and G, and T is replaced by U in an RNA. One of skill will recognize that non-naturally occurring bases be included in polynucleotides, as well.

The polynucleotide of the present invention can encode multiple peptides of the present invention with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage S L & Iyer R P, Tetrahedron 1992, 48: 2223-311; Matthes et al., EMBO J. 1984, 3: 801-5.

Vectors containing the polynucleotide of the present invention and host cells harboring the vectors are also included in the present invention.

### V. Exosomes

The present invention further provides intracellular vesicles, referred to as exosomes, that present complexes formed between the peptides of this invention and HLA antigens on their surface. Exosomes can be prepared, for example, using the methods detailed in Japanese Patent Application Kohyo Publications Nos. Hei 11-510507 and WO99/03499, and can be prepared using APCs obtained from patients who are subject to treatment and/or prevention. The exosomes of this invention can be inoculated as vaccines, in a fashion similar to the peptides of this invention.

The type of HLA antigens contained in the complexes must match that of the subject requiring treatment and/or prevention. The use of the HLA-A2 type that is highly expressed among the Japanese and Caucasian is favorable for obtaining effective results, and subtypes such as HLA-A2 (A*0201 and A*0206) also find use. Typically, in the clinic, the type of HLA antigen of the patient requiring treatment is investigated in advance, which enables the appropriate selection of peptides having high levels of binding affinity to the particular antigen, or having CTL inducibility by antigen presentation. Furthermore, in order to obtain peptides having both high binding affinity and CTL inducibility, substitution, insertion, deletion and/or addition of 1, 2, or several amino acids can be performed based on the amino acid sequence of the naturally occurring IMP-3 partial peptide.

When using the HLA-A2 (A*0201) antigen for the exosome of the present invention, the peptides having the sequence selected from among of SEQ ID NOs: 1, 3, 5 and 6 find particular use.

### VI. Antigen-Presenting Cells (APCs)

The present invention also provides isolated antigen-presenting cells (APCs) that present complexes formed between HLA antigens and the peptides of this invention on its surface. The APCs that are obtained by contacting the peptides of this invention, or introducing the polynucleotides encoding the peptides of this invention in an expressible form can be derived from patients who are subject to treatment and/or prevention, and can be administered as vaccines by themselves or in combination with other drugs including the peptides of this invention, exosomes, or cytotoxic T cells.

The APCs are not limited to a particular kind of cells and include dendritic cells (DCs), Langerhans cells, macrophages, B cells, and activated T cells, which are known to present proteinaceous antigens on their cell surface so as to be recognized by lymphocytes. Since DC is a representative APC having the strongest CTL inducing action among APCs, DCs find use as the APCs of the present invention.

For example, an APC can be obtained by inducing DCs from peripheral blood monocytes and then contacting (stimulating) them with the peptides of this invention in vitro, ex vivo or in vivo. When the peptides of this invention are administered to the subjects, APCs that present the peptides of this invention are induced in the body of the subject. The phrase “inducing APC” includes contacting (stimulating) a cell with the peptides of the present invention, or nucleotides encoding such peptides, to present complexes formed between HLA antigens and the peptides of the present invention on cell's surface. Therefore, the APCs of the present invention may be obtained by collecting the APCs from the subject after administering the peptides of the present invention to the subject. Alternatively, the APCs of the present invention may be obtained by contacting APCs collected from a subject with the peptide of the present invention.

APCs of the present invention may themselves be administered to a subject for inducing immune response against cancer in the subject, for example as a vaccine. APCs of the present invention may also be administered in combination with other drugs including the peptides, exosomes or CTLs of the present invention. Ex vivo administration can include the steps of:

a: collecting APCs from a first subject;
b: contacting the APCs of step a with the peptide; and
c: administering the peptide-loaded APCs to a second subject.

The first subject and the second subject can be the same individual, or may be different individuals. Alternatively, according to the present invention, use of the peptides of the present invention for manufacturing a pharmaceutical agent or composition inducing antigen-presenting cells is provided. In addition, the present invention provides a method or process for manufacturing a pharmaceutical agent or composition for inducing antigen-presenting cells, wherein the method includes the step of admixing or formulating the peptide of the present invention with a pharmaceutically acceptable carrier. Moreover, the present invention provides a method or process for manufacturing a pharmaceutical agent or composition for treating cancers including lung cancer and esophageal cancer, wherein the method includes the step of admixing or formulating the peptide of the present invention with a pharmaceutically acceptable carrier. Further, the present invention also provides the peptides of the present invention for inducing antigen-presenting cells. The APCs obtained by step b can be administered to the subject as a vaccine. The present invention further provides the peptides for treating cancers including lung cancer and esophageal cancer.

According to an aspect of the present invention, the APCs of the present invention have a high level of CTL inducibility. In the term of “high level of CTL inducibility”, the high level is relative to the level of that of APCs contacted with no peptide or peptides which can not induce CTLs. Such APCs having a high level of CTL inducibility can be prepared by a method which includes the step of transferring genes containing polynucleotides that encode the peptides of this invention to APCs in vitro. The introduced genes can be in the form of DNAs or RNAs. Examples of methods for introduction include, without particular limitations, various methods conventionally performed in this field, such as lipofection, electroporation, and calcium phosphate method. More specifically, it can be performed as described in Cancer Res 1996, 56: 5672-7; J Immunol 1998, 161: 5607-13; J Exp Med 1996, 184: 465-72; Published Japanese Translation of International Publication No. 2000-509281. By transferring the gene into an APC, the gene undergoes transcription, translation, and such in the cell, and then the obtained protein is processed by MHC Class I or Class II, and proceeds through a presentation pathway to present the peptides.

In a preferred embodiment, the APCs of the present invention present on its surface a complex of an HLA antigen and an oligopeptide having an amino acid sequence selected from among SEQ ID NOs: 1, 3, 5 and 6. Preferably, the APCs of the present invention carry the HLA-A2 antigen on its surface. In other words, the APCs of the present invention preferably expresses the HLA-A2 antigen on its surface. Alternatively, the oligopeptide to form the complex with an HLA antigen may be a oligopeptide having an amino acid sequence selected from among SEQ ID NOs: 1, 3, 5 and 6, wherein one, two or several amino acids are substituted, inserted, deleted and/or added; for example, the second amino acid from the N-terminus may be substituted with leucine or methionine, and/or the C-terminal amino acid may be substituted with valine or leucine.

### VII. Cytotoxic T Cells (Cytotoxic T Lymphocytes:CTLs)

A cytotoxic T cell induced against any of the peptides of the present invention strengthens the immune response targeting tumor-associated endothelia in vivo and thus can be used as vaccines, in a fashion similar to the peptides per se. Thus, the present invention also provides isolated cytotoxic T cells that are specifically induced or activated by any of the present peptides.

Such cytotoxic T cells can be obtained by (1) administering the peptide of the present invention to a subject, and then collecting cytotoxic T cells from the subject, or (2) contacting (stimulating) subject-derived APCs, and CD8-positive cells, or peripheral blood mononuclear leukocytes in vitro with the peptides of the present invention and then isolating cytotoxic T cells.

The cytotoxic T cells, which have been induced by stimulation with APCs that present the peptides of this invention, can be derived from patients who are subject to treatment and/or prevention, and can be administered by themselves or in combination with other drugs including the peptides of this invention or exosomes for the purpose of regulating effects. The obtained cytotoxic T cells act specifically against target cells presenting the peptides of this invention, or for example, the same peptides used for induction. In other words, the obtained cytotoxic T cells is able to recognize (i.e., binding to) a complex formed between an HLA antigen and the peptide of the present invention on a target cell surface via its T cell receptor, and then attack the target cell to induce the death of the target cell. The target cells can be cells that endogenously express IMP-3, or cells that are transfected with the IMP-3 gene; and cells that present a peptide of this invention on the cell surface due to stimulation by the peptide can also serve as targets of activated CTL attack. In a preferred embodiment, the target cells carry the HLA-A2 antigen on its surface and present a complex formed between HLA-A2 and the peptide of the present invention on its surface.

### VIII. T Cell Receptor (TCR)

The present invention also provides a composition containing a nucleic acid sequence encoding a polypeptide that is capable of forming a subunit of a T cell receptor (TCR), and methods of using the same. The TCR subunits, alpha and beta, have the ability to form TCRs that confer specificity to T cells against tumor cells presenting IMP-3. By using the known methods in the art, the nucleic acid sequence of TCR alpha and beta chains expressed in the CTLs induced with one or more peptides of this invention can be isolated and used for constructing suitable vectors that can mediate highly efficient gene transfers into primary human lymphocytes (WO2007/032255 and Morgan R A, et al., J Immunol, 171, 3287 (2003)). For example, the PCR method is preferred to analyze the TCR. The PCR primers for the analysis can be, for example, 5′-R primers (5′-gtctaccaggcattcgcttcat-3′) as 5′ side primers (SEQ ID NO: 23) and 3-TRa-C primers (5′-tcagctggaccacagccgcagcgt-3′) specific to TCR alpha chain C region (SEQ ID NO: 24), 3-TRb-C1 primers (5′-tcagaaatcctttctcttgac-3′) specific to TCR beta chain C1 region (SEQ ID NO: 25) or 3-TRbeta-C2 primers (5′-ctagcctctggaatcctttctctt-3′) specific to TCR beta chain C2 region (SEQ ID NO: 26) as 3′ side primers, but not limited. Exemplary vectors include, but are not limited to, retroviral vectors. Advantageously, the invention provides an off-the-shelf composition allowing rapid modification of a patient's own T cells (or those of another mammal) to rapidly and easily produce modified T cells having excellent cancer cell killing properties. The derivative TCRs can bind target cells displaying the IMP-3 peptide with high avidity, and optionally mediate efficient killing of target cells presenting the IMP-3 peptide in vivo and in vitro.

The nucleic acids encoding the TCR subunits can be incorporated into suitable vectors e.g. retroviral vectors. These vectors are well known in the art. The nucleic acids or the vectors containing them usefully can be transferred into a T cell, for example, a T cell from a patient. Advantageously, the invention provides an off-the-shelf composition allowing rapid modification of a patient's own T cells (or those of another mammal) to rapidly and easily produce modified T cells having excellent cancer cell killing properties.

The specific TCR is a receptor capable of specifically recognizing a complex of a peptide of the present invention and HLA molecule, giving a T cell specific activity against the target cell when the TCR on the surface of the T cell. A specific recognition of the above complex may be confirmed by any known methods, and preferred methods include, for example, tetramer analysis using HLA molecule and peptide of the invention, and ELISPOT assay. By performing the ELISPOT assay, it can be confirmed that a T cell expressing the TCR on the cell surface recognizes a cell by the TCR, and that the signal is transmitted intracellularly. The confirmation that the above-mentioned complex can give a T cell cytotoxic activity when the complex exists on the T cell surface may also be carried out by a known method. A preferred method includes, for example, the determination of cytotoxic activity against an HLA positive target cell, such as chromium release assay.

Also, the present invention provides CTLs which are prepared by transduction with the nucleic acids encoding the TCR subunits polypeptides that bind to the IMP-3 peptide e.g. SEQ ID NOs: 1, 3, 5 and 6 in the context of HLA-A2. The transduced CTLs are capable of homing to cancer cells in vivo, and can be expanded by well known culturing methods in vitro (e.g., Kawakami et al., J. Immunol., 142, 3452-3461 (1989)). The T cells of the invention can be used to form an immunogenic composition useful in the treatment or the prevention of cancer in a patient in need of therapy or protection (WO2006/031221).

### IX. Pharmaceutical Agents or Compositions

Since IMP-3 expression is up-regulated in several cancers as compared with normal tissue, the peptides of this invention or polynucleotides encoding such peptides can be used for the treatment and/or for the prophylaxis of cancer or tumor, and/or prevention of postoperative recurrence thereof. Thus, the present invention provides a pharmaceutical agent or composition for treating and/or for preventing of cancer or tumor, and/or preventing the postoperative recurrence thereof, that includes as an active ingredient one or more of the peptides of this invention, or polynucleotides encoding the peptides. Alternatively, the present peptides can be expressed on the surface of any of the foregoing exosomes or cells, such as APCs for the use as pharmaceutical agents or composition. In addition, the aforementioned cytotoxic T cells which target any of the peptides of the present invention can also be used as the active ingredient of the present pharmaceutical agents or compositions. In the context of the present invention, the phrase “targeting a peptide” with regard to the activity of a cytotoxic T cell indicates that the cytotoxic T cell recognizes (i.e., binds to) a complex formed between an HLA antigen and a peptide on a target cell surface via its T cell receptor, and then attacks the target cell to induce the death of the target cell.

In another embodiment, the present invention also provides the use of an active ingredient selected from among:

(a) a peptide of the present invention,

(b) a nucleic acid encoding such a peptide as disclosed herein in an expressible form,

(c) an APC of the present invention, and

(d) a cytotoxic T cells of the present invention

in manufacturing a pharmaceutical composition or agent for treating cancer or tumor.

Alternatively, the present invention further provides an active ingredient selected from among:

(a) a peptide of the present invention,

(b) a nucleic acid encoding such a peptide as disclosed herein in an expressible form,

(c) an APC of the present invention, and

(d) a cytotoxic T cells of the present invention for use in treating cancer or tumor.

Alternatively, the present invention further provides a method or process for manufacturing a pharmaceutical composition or agent for treating cancer or tumor, wherein the method or process includes the step of formulating a pharmaceutically or physiologically acceptable carrier with an active ingredient selected from among:

(a) a peptide of the present invention,

(b) a nucleic acid encoding such a peptide as disclosed herein in an expressible form,

(c) an APC of the present invention, and

(d) a cytotoxic T cells of the present invention

as active ingredients.

In another embodiment, the present invention also provides a method or process for manufacturing a pharmaceutical composition or agent for treating cancer or tumor, wherein the method or process includes the step of admixing an active ingredient with a pharmaceutically or physiologically acceptable carrier, wherein the active ingredient is selected from among:

(a) a peptide of the present invention,

(b) a nucleic acid encoding such a peptide as disclosed herein in an expressible form,

(c) an APC of the present invention, and

(d) a cytotoxic T cells of the present invention.

Alternatively, the pharmaceutical composition or agent of the present invention may be used for either or both the prophylaxis of cancer or tumor and prevention of post-operative recurrence thereof.

The pharmaceutical agents or compositions of the present invention can be used to treat and/or prevent cancers or tumors, and/or prevention of postoperative recurrence thereof in subjects or patients including human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal.

According to the present invention, oligopeptides having an amino acid sequence selected from among SEQ ID NOs: 1, 3, 5 and 6 have been found to be HLA-A2-restricted epitope peptides that can induce potent and specific immune response. Therefore, the present pharmaceutical agents or compositions which include any of these oligopeptides having the amino acid sequences of SEQ ID NOs: 1, 3, 5 or 6 are particularly suited for the administration to subjects whose HLA antigen is HLA-A2. As used herein, “subjects whose HLA antigen is HLA-A2” means subjects who possess the HLA-A2 gene homozygously or heterozygously and HLA-A2 is expressed in cells of the subjects as an HLA antigen. In other words, subjects are HLA-A2 positive. The same applies to pharmaceutical agents or compositions which include polynucleotides encoding any of these oligopeptides.

Cancers or tumors to be treated by the pharmaceutical agents or compositions of the present invention are not limited and include all kinds of cancers or tumors wherein IMP-3 is involved, including for example, lung cancer and esophageal cancer. In particular, the pharmaceutical agents or compositions of the present invention are preferably applied to pancreatic cancer.

The present pharmaceutical agents or compositions can contain in addition to the aforementioned active ingredients, other peptides which have the ability to induce CTLs against cancerous cells, other polynucleotides encoding the other peptides, other cells that present the other peptides, or such. Herein, the other peptides that have the ability to induce CTLs against cancerous cells are exemplified by cancer specific antigens (e.g., identified TAAs), but are not limited thereto.

If needed, the pharmaceutical agents or compositions of the present invention can optionally include other therapeutic substances as an active ingredient, so long as the substance does not inhibit the antitumoral effect of the active ingredient, e.g., any of the present peptides. For example, formulations can include anti-inflammatory agents, pain killers, chemotherapeutics, and the like. In addition to including other therapeutic substances in the medicament itself, the medicaments of the present invention can also be administered sequentially or concurrently with the one or more other pharmacologic agents or compositions. The amounts of medicament and pharmacologic agent or compositions depend, for example, on what type of pharmacologic agent(s) or compositions(s) is/are used, the disease being treated, and the scheduling and routes of administration.

It should be understood that in addition to the ingredients particularly mentioned herein, the pharmaceutical agents or compositions of this invention can include other agents or compositions conventional in the art having regard to the type of formulation in question.

In one embodiment of the present invention, the present pharmaceutical agents or compositions can be included in articles of manufacture and kits containing materials useful for treating the pathological conditions of the disease to be treated, e.g., cancer. The article of manufacture can include a container of any of the present pharmaceutical agents or compositions with a label. Suitable containers include bottles, vials, and test tubes. The containers can be formed from a variety of materials, such as glass or plastic. The label on the container should indicate the agent or compositions are used for treating or prevention of one or more conditions of the disease. The label can also indicate directions for administration and so on.

In addition to the container described above, a kit including a pharmaceutical agent or compositions of the present invention can optionally further include a second container housing a pharmaceutically-acceptable diluent. It can further include other materials desirable from a commercial and user standpoint, including other buffers, diluents, filters, needles, syringes, and package inserts with instructions for use.

The pharmaceutical agents or compositions can, if desired, be presented in a pack or dispenser device which can contain one or more unit dosage forms containing the active ingredient. The pack can, for example, include metal or plastic foil, such as a blister pack. The pack or dispenser device can be accompanied by instructions for administration.

In another embodiment of the present invention, the peptides of the present invention may also be administered in the form of a pharmaceutically acceptable salt. Preferable examples of the salts include salts with an alkali metal, salts with a metal, salts with an organic base, salts with an organic acid and salts with an inorganic acid.

(1) Pharmaceutical Agents or Compositions Containing the Peptides as the Active Ingredient

The peptides of this invention can be administered directly as a pharmaceutical agent or compositions, or if necessary, may be formulated by conventional formulation methods. In the latter case, in addition to the peptides of this invention, carriers, excipients, and such that are ordinarily used for drugs can be included as appropriate without particular limitations. Examples of such carriers are sterilized water, physiological saline, phosphate buffer, culture fluid and such. Furthermore, the pharmaceutical agents or compositions can contain as necessary, stabilizers, suspensions, preservatives, surfactants and such. The pharmaceutical agents or compositions of this invention can be used for anticancer purposes.

The peptides of this invention can be prepared as a combination, composed of two or more of peptides of the present invention, to induce CTLs in vivo. The peptide combination can take the form of a cocktail or can be conjugated to each other using standard techniques. For example, the peptides can be chemically linked or expressed as a single fusion polypeptide sequence. The peptides in the combination can be the same or different. By administering the peptides of this invention, the peptides are presented at a high density by the HLA antigens on APCs, then CTLs that specifically react toward the complex formed between the displayed peptide and the HLA antigen are induced. Alternatively, APCs that present any of the peptides of this invention on their cell surface, which may be obtained by stimulating APCs (e.g., DCs) derived from a subject with the peptides of this invention, may be administered to the subject, and as a result, CTLs are induced in the subject and aggressiveness towards the cancer cells, such as lung cancer and esophageal cancer cells, can be increased.

The pharmaceutical agents or compositions for the treatment and/or prevention of cancer or tumor, which include a peptide of this invention as the active ingredient, can also include an adjuvant known to effectively establish cellular immunity. Alternatively, the pharmaceutical agents or compositions can be administered with other active ingredients or administered by formulation into granules. An adjuvant refers to a compound that enhances the immune response against the protein when administered together (or successively) with the protein having immunological activity. Adjuvants contemplated herein include those described in the literature (Clin Microbiol Rev 1994, 7: 277-89). Example of suitable adjuvants include, but are not limited to, aluminum phosphate, aluminum hydroxide, alum, cholera toxin, salmonella toxin, and such, but are not limited thereto.

Furthermore, liposome formulations, granular formulations in which the peptide is bound to few-micrometers diameter beads, and formulations in which a lipid is bound to the peptide may be conveniently used.

In another embodiment of the present invention, the peptides of the present invention may also be administered in the form of a pharmaceutically acceptable salt. Preferable examples of the salts include salts with an alkali metal, salts with a metal, salts with an organic base, salts with an organic acid and salts with an inorganic acid. As used herein, “pharmaceutically acceptable salt” refers to those salts which retain the biological effectiveness and properties of the compound and which are obtained by reaction with inorganic acids or bases such as hydrochloric acid, hydrobromic acid, sulfuric acid, nitric acid, phosphoric acid, methanesulfonic acid, ethanesulfonic acid, p-toluenesulfonic acid, salicylic acid and the like. Examples of preferred salts include salts with an alkali metal, salts with a metal, salts with an organic base, salts with an organic acid and salts with an inorganic acid.

In some embodiments, the pharmaceutical agents or compositions of the present invention may further include a component which primes CTLs. Lipids have been identified as agents or compositions capable of priming CTLs in vivo against viral antigens. For example, palmitic acid residues can be attached to the epsilon- and alpha-amino groups of a lysine residue and then linked to a peptide of the present invention. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinlyseryl-serine (P3CSS) can be used to prime CTLs when covalently attached to an appropriate peptide (see, e.g., Deres et al., Nature 1989, 342: 561-4).

The method of administration can be oral, intradermal, subcutaneous, intravenous injection, or such, and systemic administration or local administration to the vicinity of the targeted sites. The administration can be performed by single administration or boosted by multiple administrations. The dose of the peptides of this invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once in a few days to few months. One skilled in the art can appropriately select a suitable dose.

(2) Pharmaceutical Agents or Compositions Containing Polynucleotides as the Active Ingredient

The pharmaceutical agents or compositions of the present invention can also contain nucleic acids encoding the peptides disclosed herein in an expressible form. Herein, the phrase “in an expressible form” means that the polynucleotide, when introduced into a cell, will be expressed in vivo as a polypeptide that induces anti-tumor immunity. In an exemplified embodiment, the nucleic acid sequence of the polynucleotide of interest includes regulatory elements necessary for expression of the polynucleotide. The polynucleotide(s) can be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The peptides of the present invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another example is BCG (Bacille Calmette Guerin). BCG vectors are described in Stover et al., Nature 1991, 351: 456-60. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide into a subject can be either direct, in which case the subject is directly exposed to a polynucleotide-carrying vector, or indirect, in which case, cells are first transformed with the polynucleotide of interest in vitro, then the cells are transplanted into the subject. Theses two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11 (5): 155-215. Methods commonly known in the art of recombinant DNA technology which can also be used for the present invention are described in eds. Ausubel et al., Current Protocols in Molecular Biology, John Wiley & Sons, NY, 1993; and Krieger, Gene Transfer and Expression, A Laboratory Manual, Stockton Press, NY, 1990.

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides of this invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once every a few days to once every few months. One skilled in the art can appropriately select the suitable dose.

### X. Methods Using the Peptides, Exosomes, APCs and CTLs

The peptides of the present invention and polynucleotides encoding such peptides can be used for inducing APCs and CTLs, as well as for inducing immune response against cancer or tumor. The exosomes and APCs of the present invention can be also used for inducing CTLs, as well as for inducing immune response against cancer or tumor. The peptides, polynucleotides, exosomes and APCs can be used in combination with any other compounds so long as the compounds do not inhibit their CTL inducibility. Thus, any of the aforementioned pharmaceutical agents or compositions of the present invention can be used for inducing CTLs, and in addition thereto, those including the peptides and polynucleotides can be also be used for inducing APCs as discussed below. Further, the CTLs of the present invention can also be used for inducing immune response against cancer or tumor.

(1) Method of Inducing Antigen-Presenting Cells (APCs)

The present invention provides methods of inducing APCs using the peptides of this invention or polynucleotides encoding the peptides. The induction of APCs can be performed as described above in section “VI. Antigen-presenting cells”. This invention also provides a method for inducing APCs having a high level of CTL inducibility, the induction of which has been also mentioned under the item of “VI. Antigen-presenting cells”, supra.

Preferably, the methods for inducing APCs include at least one step selected from among:

a: contacting APCs with a peptide of the present invention, and
b: introducing a polynucleotide encoding a polypeptide of the present invention in an expressible form into APCs.

Such methods for inducing APCs are preferably performed in vitro or ex vivo. To perform the methods in vitro or ex vivo, APCs may be obtained from the subject to be treated or others whose HLA antigens are the same as the subject to be treated. In a preferred embodiment, APCs induced by the present methods carry the HLA-A2 antigens on their surface.

(2) Method of Inducing CTLs

The present invention also provides methods for inducing CTLs using the peptides of this invention, polynucleotides encoding the peptides, or exosomes or APCs presenting the peptides.

The present invention also provides methods for inducing CTLs using a polynucleotide encoding a polypeptide that is capable of forming a T cell receptor (TCR) subunit recognizing (i.e., binding to) a complex of the peptides of the present invention and HLA antigens. Preferably, the methods for inducing CTLs include at least one step selected from among:

a: contacting a CD8-positive T cell with an antigen-presenting cell and/or an exosome that presents on its surface a complex of an HLA antigen and a peptide of the present invention, and

b: introducing a polynucleotide encoding a polypeptide that is capable of forming a TCR subunit recognizing a complex of a peptide of the present invention and an HLA antigen into a CD8 positive T cell.

When the peptides of the present invention are administered to a subject, CTLs are induced in the body of the subject, and the strength of the immune response targeting the tumor-associated endothelia is enhanced. Alternatively, the peptides and polynucleotides encoding the peptides can be used for an ex vivo therapeutic method, in which subject-derived APCs, and CD8-positive cells, or peripheral blood mononuclear leukocytes are contacted (stimulated) with the peptides of this invention in vitro, and after inducing CTLs, the activated CTL cells are returned to the subject. For example, the method can include steps of:

a: collecting APCs from subject,

b: contacting the peptide with the APCs of step a,

c: mixing the APCs of step b with CD⁸⁺ T cells, and co-culturing for inducing CTLs, and

d: collecting CD⁸⁺ T cells from the co-culture of step c.

Alternatively, according to the present invention, use of the peptides of this invention for manufacturing a pharmaceutical agent or composition inducing CTLs is provided. In addition, the present invention provides a method or process for manufacturing a pharmaceutical agent or composition inducing CTLs, wherein the method includes the step of admixing or formulating the peptide of the present invention with a pharmaceutically acceptable carrier. Further, the present invention also provides the peptide of the present invention for inducing CTLs.

The CD8⁺ T cells having cytotoxic activity obtained by step d can be administered to the subject as a vaccine. The APCs to be mixed with the CD8⁺ T cells in above step c can also be prepared by transferring genes coding for the present peptides into the APCs as detailed above in section “VI. Antigen-presenting cells”; but are not limited thereto. Accordingly, any APCs or exosomes which effectively presents the present peptides to the T cells can be used for the present method.

(3) Method of Inducing Immune Response

The present invention further provides methods for inducing an immune response against cancer, such as lung cancer and esophageal cancer, in a subject. The methods include the administration of a vaccine one the present invention, which includes:

(a) one or more oligopeptides of the present invention, or an immunologically active fragment thereof;

(b) one or more polynucleotides encoding the oligopeptides or the immunologically active fragment of (a);

(c) one or more isolated CTLs of the present invention;

(d) one or more isolated antigen-presenting cells of the present invention; or

(e) one or more T cells isolated and transformed with a TCR encoding gene.

In the context of the present invention, cancer overexpressing IMP-3 can be treated with these active ingredients. Examples of such cancers include, but are not limited to, lung cancer and esophageal cancer. Accordingly, prior to the administration of the vaccines or pharmaceutical compositions containing the active ingredients, it is preferable to confirm whether the expression level of IMP-3 in the cancer cells or tissues to be treated is enhanced as compared with normal cells of the same organ. Thus, in one embodiment, the present invention provides a method for treating cancer (over)expressing IMP-3, which method may include the steps of:

i) determining the expression level of IMP-3 in cancer cells or tissue(s) obtained from a subject with the cancer to be treated;

ii) comparing the expression level of IMP-3 with normal control; and

iii) administrating at least one component selected from among (a) to (d) described above to a subject with cancer overexpressing IMP-3 compared with normal control. Alternatively, the present invention may provide a vaccine or pharmaceutical composition that includes at least one component selected from among (a) to (d) described above, for use in administrating to a subject having cancer overexpressing IMP-3. In other words, the present invention further provides a method for identifying a subject to be treated with a IMP-3 polypeptide of the present invention, such method including the step of determining an expression level of IMP-3 in subject-derived cancer cells or tissue(s), wherein an increase of the level compared to a normal control level of the gene indicates that the subject has cancer which may be treated with the IMP-3 polypeptide of the present invention. Methods of treating cancer of the present invention are described in more detail below.

Any subject-derived cell or tissue can be used for the determination of IMP-3 expression so long as it includes the objective transcription or translation product of IMP-3. Examples of suitable samples include, but are not limited to, bodily tissues and fluids, such as blood, sputum and urine. Preferably, the subject-derived cell or tissue sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the subjected-derived sample.

A subject to be treated by the present method is preferably a mammal. Exemplary mammals include, but are not limited to, e.g., human, non-human primate, mouse, rat, dog, cat, horse, and cow.

According to the present invention, the expression level of IMP-3 in cancer cells or tissues obtained from a subject is determined. The expression level can be determined at the transcription product level, using methods known in the art. For example, the mRNA of IMP-3 may be quantified using probes by hybridization methods (e.g., Northern hybridization). The detection may be carried out on a chip or an array. The use of an array is preferable for detecting the expression level of IMP-3. Those skilled in the art can prepare such probes utilizing the sequence information of IMP-3. For example, the cDNA of IMP-3 may be used as the probes. If necessary, the probes may be labeled with a suitable label, such as dyes, fluorescent substances and isotopes, and the expression level of the gene may be detected as the intensity of the hybridized labels.

Furthermore, the transcription product of IMP-3 (e.g., SEQ ID NO: 21) may be quantified using primers by amplification-based detection methods (e.g., RT-PCR). Such primers may be prepared based on the available sequence information of the gene.

Specifically, a probe or primer used for the present method hybridizes under stringent, moderately stringent, or low stringent conditions to the mRNA of IMP-3. As used herein, the phrase “stringent (hybridization) conditions” refers to conditions under which a probe or primer will hybridize to its target sequence, but not to other sequences. Stringent conditions are sequence-dependent and will be different under different circumstances. Specific hybridization of longer sequences is observed at high temperatures than shorter sequences. Generally, the temperature of a stringent condition is selected to be about 5 degree Centigrade lower than the thermal melting point (Tm) for a specific sequence at a defined ionic strength and pH. The Tm is the temperature (under a defined ionic strength, pH and nucleic acid concentration) at which 50% of the probes complementary to their target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

The probes or primers may be of specific sizes. The sizes may range from at least 10 nucleotides, at least 12 nucleotides, at least 15 nucleotides, at least 20 nucleotides, at least 25 nucleotides, at least 30 nucleotides and the probes and primers may range in size from 5-10 nucleotides, 10-15 nucleotides, 15-20 nucleotides, 20-25 nucleotides and 25-30 nucleotides.

Alternatively, the translation product may be detected for the diagnosis of the present invention. For example, the quantity of IMP-3 protein (SEQ ID NO: 22) may be determined. Methods for determining the quantity of the protein as the translation product include immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment or modified antibody retains the binding ability to the IMP-3 protein. Methods to prepare these kinds of antibodies for the detection of proteins are well known in the art, and any method may be employed in the present invention to prepare such antibodies and equivalents thereof.

As another method to detect the expression level of IMP-3 gene based on its translation product, the intensity of staining may be measured via immunohistochemical analysis using an antibody against the IMP-3 protein. Namely, in this measurement, strong staining indicates increased presence/level of the protein and, at the same time, high expression level of IMP-3 gene.

The expression level of a target gene, e.g., the IMP-3 gene, in cancer cells can be determined to be increased if the level increases from the control level (e.g., the level in normal cells) of the target gene by, for example, 10%, 25%, or 50%; or increases to more than 1.1 fold, more than 1.5 fold, more than 2.0 fold, more than 5.0 fold, more than 10.0 fold, or more.

The control level may be determined at the same time as the cancer cells, by using a sample(s) previously collected and stored from a subject/subjects whose disease state(s) (cancerous or non-cancerous) is/are known. In addition, normal cells obtained from non-cancerous regions of an organ that has the cancer to be treated may be used as normal control. Alternatively, the control level may be determined by a statistical method based on the results obtained by analyzing previously determined expression level(s) of IMP-3 gene in samples from subjects whose disease states are known. Furthermore, the control level can be derived from a database of expression patterns from previously tested cells. Moreover, according to an aspect of the present invention, the expression level of IMP-3 gene in a biological sample may be compared to multiple control levels determined from multiple reference samples. It is preferred to use a control level determined from a reference sample derived from a tissue type similar to that of the subject-derived biological sample. Moreover, it is preferred to use the standard value of the expression levels of IMP-3 gene in a population with a known disease state. The standard value may be obtained by any method known in the art. For example, a range of mean+/−2 S.D. or mean+/−3 S.D. may be used as the standard value.

In the context of the present invention, a control level determined from a biological sample that is known to be non-cancerous is referred to as a “normal control level”. On the other hand, if the control level is determined from a cancerous biological sample, it is referred to as a “cancerous control level”. Difference between a sample expression level and a control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

When the expression level of IMP-3 gene is increased as compared to the normal control level, or is similar/equivalent to the cancerous control level, the subject may be diagnosed with cancer to be treated.

More specifically, the present invention provides a method of (i) diagnosing whether a subject has the cancer to be treated, and/or (ii) selecting a subject for cancer treatment, which method includes the steps of:

a) determining the expression level of IMP-3 in cancer cells or tissue(s) obtained from a subject who is suspected to have the cancer to be treated;

b) comparing the expression level of IMP-3 with a normal control level;

c) diagnosing the subject as having the cancer to be treated, if the expression level of IMP-3 is increased as compared to the normal control level; and

d) selecting the subject for cancer treatment, if the subject is diagnosed as having the cancer to be treated, in step c).

Alternatively, such a method includes the steps of:

a) determining the expression level of IMP-3 in cancer cells or tissue(s) obtained from a subject who is suspected to have the cancer to be treated;

b) comparing the expression level of IMP-3 with a cancerous control level;

c) diagnosing the subject as having the cancer to be treated, if the expression level of IMP-3 is similar or equivalent to the cancerous control level; and

d) selecting the subject for cancer treatment, if the subject is diagnosed as having the cancer to be treated, in step c).

The present invention also provides a kit for determining a subject suffering from cancer that can be treated with the IMP-3 polypeptide of the present invention, which may also be useful in assessing and/or monitoring the efficacy of a particular cancer therapy, more particularly a cancer immunotherapy. Illustrative examples of suitable cancers include, but are not limited to, lung cancer and esophageal cancer. More particularly, the kit preferably includes at least one reagent for detecting the expression of the IMP-3 gene in a subject-derived cancer cell, such reagent being selected from the group of:

(a) a reagent for detecting mRNA of the IMP-3 gene;

(b) a reagent for detecting the IMP-3 protein; and

(c) a reagent for detecting the biological activity of the IMP-3 protein.

Examples of reagents suitable for detecting mRNA of the IMP-3 gene include nucleic acids that specifically bind to or identify the IMP-3 mRNA, such as oligonucleotides that have a complementary sequence to a portion of the IMP-3 mRNA. These kinds of oligonucleotides are exemplified by primers and probes that are specific to the IMP-3 mRNA. These kinds of oligonucleotides may be prepared based on methods well known in the art. If needed, the reagent for detecting the IMP-3 mRNA may be immobilized on a solid matrix. Moreover, more than one reagent for detecting the IMP-3 mRNA may be included in the kit.

On the other hand, examples of reagents suitable for detecting the IMP-3 protein include antibodies to the IMP-3 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment or modified antibody retains the binding ability to the IMP-3 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of the antibodies to their targets are well known in the art, and any labels and methods may be employed for the present invention. Moreover, more than one reagent for detecting the IMP-3 protein may be included in the kit.

The kit may contain more than one of the aforementioned reagents. For example, tissue samples obtained from subjects without cancer or suffering from cancer, may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

As an embodiment of the present invention, when the reagent is a probe against the IMP-3 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of a test sample, the number of sites displaying a detectable signal provides a quantitative indication of the amount of IMP-3 mRNA present in the sample. The detection sites may be configured in any suitably detectable shape and are typically in the shape of a bar or dot spanning the width of a test strip.

The kit of the present invention may further include a positive control sample or IMP-3 standard sample. The positive control sample of the present invention may be prepared by collecting IMP-3 positive samples and then assaying their IMP-3 levels. Alternatively, a purified IMP-3 protein or polynucleotide may be added to cells that do not express IMP-3 to form the positive sample or the IMP-3 standard sample. In the present invention, purified IMP-3 may be a recombinant protein. The IMP-3 level of the positive control sample is, for example, more than the cut off value.

The following examples are presented to illustrate the present invention and to assist one of ordinary skill in making and using the same. The examples are not intended in any way to otherwise limit the scope of the invention.

### EXAMPLES

### Materials and Methods

Mice

Human leukocyte antigen (HLA)-A2 Transgenic (Tg) mice; H-2D^{b} and beta2m double knockout mice introduced with a human beta2m-HLA-A2.1 (HLA-A*0201, alpha 1, alpha 2)-H-2D^{b} (alpha 3 transmembrane cytoplasmic) monochain construct gene were generated in the Department SIDA-Retrovirus, Unite d'Immunite Cellulaire Antivirale, Institute Pasteur, France and kindly provided by Dr. F. A. Lemonnier. The mice were maintained at the Center for Animal Resources and Development of Kumamoto University and they were handled in accordance with the animal care guidelines of Kumamoto University.

Cell Lines

PANC1, A549, Lu99, MCF7, SW620, SKHep1 and T2, TAP-deficient and HLA-A2 (A*0201)-positive cell line, were purchased from Riken Cell Bank, Tsukuba, Japan. The expression of IMP-3 was determined by reverse transcription-polymerase chain reaction analysis.

Blood Samples

The researches done by using peripheral blood mononuclear cells (PBMCs) isolated from HLA-A2-positive donors were approved by the Institutional Review Board of Kumamoto University, Kumamoto, Japan. Blood samples of 4 patients with lung cancer, designated patient 1, patient 3 and patient 4, patient 14 and patient 103, were obtained during routine diagnostic procedures after obtaining formal written informed consents by the patients in Kumamoto University Hospital. Blood samples were also obtained from HLA-A2 (A*0201)-positive healthy donors, designated donor-1, donor-2 and donor-3, after receiving the written informed consent. All samples were anonymized, numbered at random, and stored at −80 degrees C. until use.

Candidate Selection of Peptides Derived from IMP-3

Peptides derived from IMP-3 that can possibly bind to HLA-A2 (A*0201) molecule were predicted using binding prediction software “BIMAS” (http://www-bimas.cit.nih.gov/molbio/hla_bind) (Parker et al., J Immunol 1994, 152 (1): 163-75, Kuzushima et al., Blood 2001, 98 (6): 1872-81). These peptides and the HLA-A2 (A*0201)-restricted HIV peptide (SLYNTYATL) were synthesized by American Peptide Company, Sunnyvale, Calif., USA with the purity >95%.

Induction of IMP-3-Reactive Mouse CTLs

HLA-A2 Tg mice were immunized with 5×10⁵ syngeneic bone marrow derived dendritic cells (BM-DCs) pulsed with candidate peptides in vivo on day 7 and 14. On day 21, CD4⁻ spleen cells isolated from the immunized mice were stimulated with BM-DCs pulsed with each peptide for 6 days. IFN-gamma production was detected by an enzyme-linked immunospot (ELISPOT) assay.

Induction of IMP-3-Reactive Human CTLs

PBMCs from heparinized blood of HLA-A2 (A*0201)-positive donors were isolated by means of Ficoll-Conray density gradient centrifugation to generate peripheral monocyte-derived DCs. The DCs were pulsed with 20 micro-g/mL of the candidate peptides in the presence of 4 micro-g/mL beta2-microglobulin (Sigma-Aldrich, St. Louis, Mo., USA) for 2 hours at 37 degrees C. in AIM-V (Invitrogen Japan, Tokyo, Japan) containing 2% heat-inactivated autologous plasma. The cells were then irradiated (40 Gy) and incubated with the CD8⁺ T cells. These cultures were set up in 24-well plates, each well contained 1×10⁵ peptide-pulsed DCs, 2×10⁶ CD8⁺ T cells and 5 ng/mL human recombinant IL-7 (Wako, Osaka, Japan) in 2 mL AIM-V with 2% autologous plasma. After 2 days, these cultures were supplemented with human recombinant IL-2 (PeproTech, Rocky Hill, N.J., USA) to a final concentration of 20 IU/mL. Two additional weekly stimulations with peptide-loaded autologous DCs, using the same procedure, were carried out on days 7 and 14. Six days after the last stimulation, the antigen-specific responses of the induced CTLs were investigated by IFN-gamma ELISPOT assay and ⁵¹Cr release assay. For IFN-gamma ELISPOT assay, CTLs (1×10⁵ cells/well) were stimulated with T2 (1×10⁴/well) pulsed with cognate peptides or the irrelevant HIV peptide. For ⁵¹Cr release assay, CTLs were co-cultured with peptide-pulsed T2 cells or cancer cells as a target cells (5×10³/well) at the indicated effector/target ratio and a standard ⁵¹Cr release assay was done as described previously (Komori H et al., Clin Cancer Res. 2006 May 1; 12 (9):2689-97).

Analysis of CD107a (LAMP-1; Lysosomal-Associated Membrane Protein-1) Exposure on the Cell Surface of CTLs

The exposure of CD107a on the cell surface of the CTLs after antigen stimulation was detected by anti-CD107a antibody. IMP-3 peptide-specific CTLs were stimulated with cognate peptide or irrelevant HIV peptide in the presence of FITC-conjugated anti-CD107a mAb or Mouse IgG1 as a control. These CTLs were cultured for 5 hours at 37 degrees C. and were subsequently stained with PE conjugated anti-human CD8 mAb. All peptides were used at a final concentration of 1 microgram/ml. Events shown are gated for CD8⁺ T cells.

Inhibition of CTL Responses by Anti-HLA-Class I Monoclonal Antibody

The inhibition of HLA-class I was done as described previously (Komori H et al., Clin Cancer Res. 2006 May 1; 12(9):2689-97). Specifically, after Lu99 target cells were incubated with anti-HLA class I mAb (W6/32, IgG2a) or anti-HLA-DR mAb (HLA-class II mAb) (H-DR-1, IgG2a), respectively, for 1 hour, Lu99 cells were co-cultured with CTLs derived from lung cancer patients by stimulation with cognate peptides.

Statistical Analysis

The two-tailed Student's t-test was used to evaluate the statistical significance of differences in the data obtained by IFN-gamma ELISPOT assay. A value of P<0.05 was considered to be significant. The statistical analysis was performed using a commercial statistical software package (SPSS for Windows, version 11.0, Chicago, Ill., USA).

Results

Prediction of HLA-A2 Binding Peptides Derived from IMP-3

Table 1 shows the HLA-A2 (A*0201) binding peptides of IMP-3 in order of highest binding affinity (Table 1). A total of 20 peptides with potential HLA-A2 (A*0201) binding capacity were selected.

TABLE 1
HLA-A2 (A*0201) binding peptides
derived from IMP-3

| SEQ | | | HLA-A2 |
|---|---|---|---|
| ID | | Amino acid | Binding |
| NO. | Position | sequence | Score |
| 1 | 199-207 | RLLVPTQFV | 1415.4 |
| 2 | 280-288 | KILAHNNFV | 681.2 |
| 3 | 552-560 | KIQEILTQV | 315.6 |
| 4 | 92-100 | LQWEVLDSL | 141.2 |
| 5 | 26-34 | KIPVSGPFL | 56.5 |
| 6 | 515-523 | NLSSAEVVV | 28.5 |
| 7 | 223-231 | KQTQSKIDV | 24.7 |
| 8 | 367-375 | GLNLNALGL | 21.4 |
| 9 | 99-107 | SLLVQYGVV | 20.6 |
| 10 | 374-382 | GLFPPTSGM | 18.4 |
| 11 | 423-431 | KQGQHIKQL | 17.4 |
| 12 | 143-151 | QLENFTLKV | 16.9 |
| 13 | 407-415 | TVHLFIPAL | 16.3 |
| 14 | 502-510 | VIGKGGKTV | 16.3 |
| 15 | 263-271 | IMHKEAQDI | 12.8 |
| 16 | 429-437 | KQLSRFAGA | 12.4 |
| 17 | 105-113 | GVVESCEQV | 12.2 |
| 18 | 513-521 | LQNLSSAEV | 12.0 |
| 19 | 409-417 | HLFIPALSV | 8.8 |
| 20 | 321-329 | YNPERTITV | 8.6 |

Induction of IMP-3-Reactive and HLA-A2-Restricted CTLs Using HLA-A2 Transgenic Mice

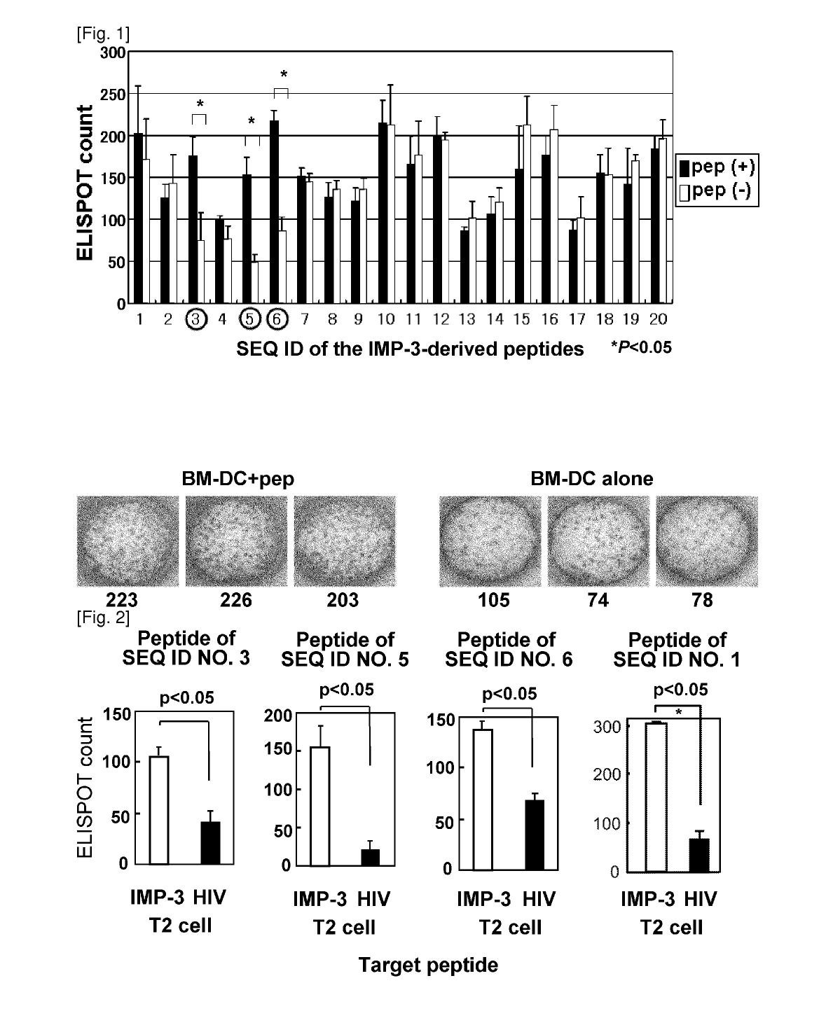

To test which of the peptides can induce peptide-reactive cytotoxic T lymphocytes (CTLs), CD4⁻ spleen cells from HLA-A2 (A*0201) transgenic (Tg) mice immunized twice with 9-mer peptides were stimulated in vitro as described in Materials and Methods. It was discovered that the CD4⁻ spleen cells stimulated with IMP-3-552-560 (SEQ ID NO: 3), IMP-3-26-34 (SEQ ID NO: 5) and IMP-3-515-523 (SEQ ID NO: 6) peptides produced IFN-gamma in response to syngeneic BM-DCs pulsed with cognate peptides. Compared with IFN-gamma production against BM-DCs alone, these CD4⁻ spleen cells recognized antigen presenting cells and produced IFN-gamma (P<0.05) (FIG. 1). These results showed that IMP-3-552-560 (SEQ ID NO: 3), IMP-3-26-34 (SEQ ID NO: 5) and IMP-3-515-523 (SEQ ID NO: 6) peptides could induce CTLs having the potent activity of IFN-gamma production in the HLA-A2 Tg mice.

Induction of IMP-3-Reactive and HLA-A2-Restricted Human CTLs

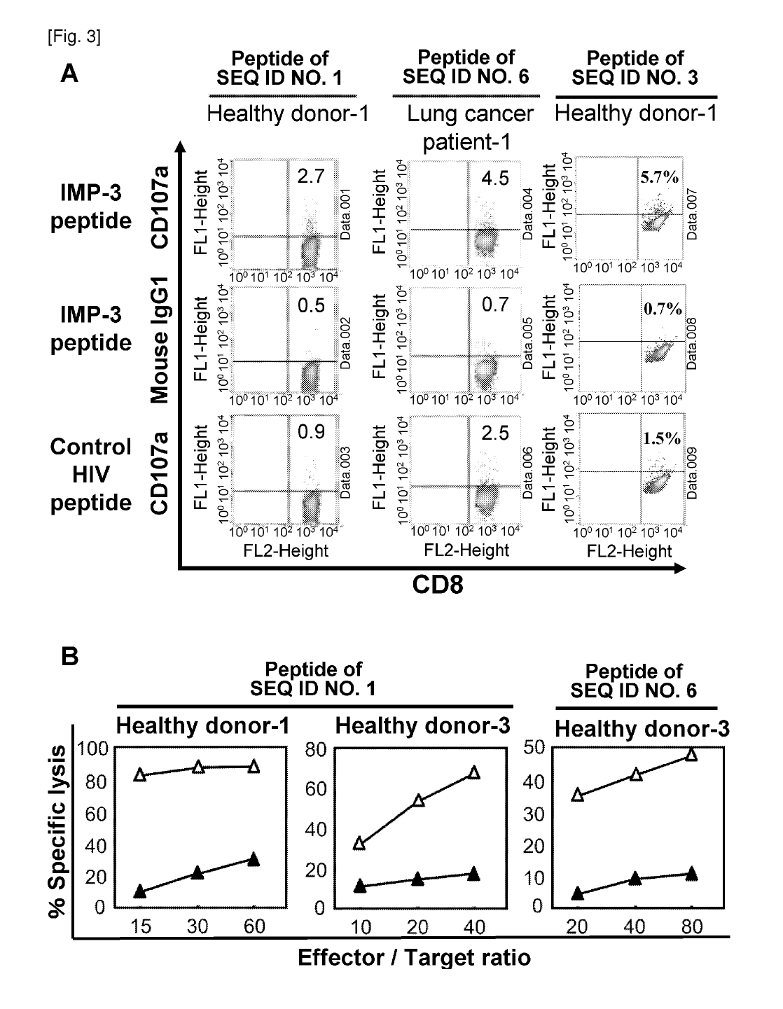

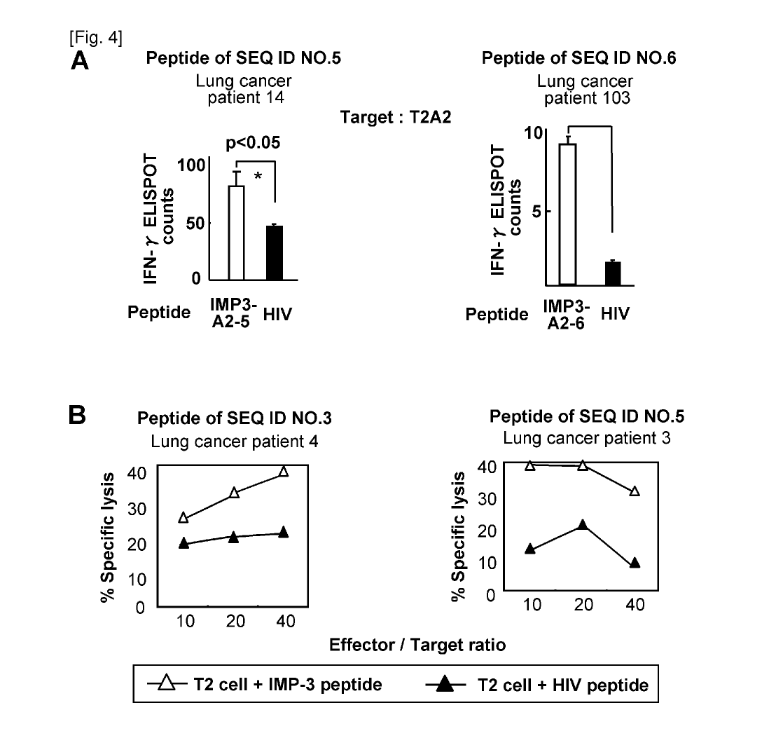

IMP-3-reactive CTLs were generated from PBMCs of HLA-A2 (A*0201)-positive healthy donor-1 by the stimulation of PBMCs with IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides. The production of IFN-gamma against peptide-pulsed T2 cells was examined by IFN-gamma ELISPOT assay. The CTLs exhibited potent IFN-gamma production against T2 cells pulsed with cognate IMP-3 peptides with a significant difference compared to that against T2 cells pulsed with irrelevant HIV peptide (P<0.05) (FIG. 2). These results indicate that IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides could induce human CTLs specific to these peptide. Furthermore, the exposure of CD107a on the cell surface of IMP-3-199-207 (SEQ ID NO: 1), IMP-3-552-560 (SEQ ID NO: 3) and IMP-3-515-523 (SEQ ID NO: 6) peptide specific CTLs was analyzed to examine the cytolytic activity. CTLs were stimulated with IMP-3-552-560 (SEQ ID NO: 3) peptide and stained with anti-CD107a mAb or mouse IgG as a control (FIG. 3A). CTLs stimulated with irrelevant HIV peptide were also stained with anti-CD107a mAb (right panel). The CD8⁺/CD107a+ cells were detected in 5.7% of all CD8⁺ cells by stimulation with IMP-3-552-560 (SEQ ID NO: 3) peptide (left panel). As non-specific signal, staining with mouse IgG was detected in 0.7% of the cells and CD8⁺/CD107a⁺ cells were detected in 1.5% of the cells stimulated with HIV peptide as a negative control (middle and right panels). As CD107a is not usually presented on the cell surface of CTLs but are exposed only during active degranulation (Betts M et al., J Immunol Methods. 2003 Oct. 1; 281 (1-2):65-78), this result indicates that CTLs exhibits a cytotoxic activity in response to IMP-3-199-207 (SEQ ID NO: 1), IMP-3-552-560 (SEQ ID NO: 3) peptide and IMP-3-515-523 (SEQ ID NO: 6). The cytotoxic activity against peptide-pulsed T2 cells was examined by ⁵¹Cr-release assays (FIG. 3B). The CTLs induced from the PBMCs of healthy donors exhibited cytotoxic activity to the T2 cells pulsed with IMP-3-199-207 (SEQ ID NO: 1) or IMP-3-515-523 (SEQ ID NO: 6) peptide, but not to the T2 cells pulsed with an irrelevant HIV-A2 peptide. These results indicate that these CTLs have a peptide-specific cytotoxicity.

Induction of IMP-3-Reactive and HLA-A2-Restricted CTLs from PBMCs of Lung Cancer Patients

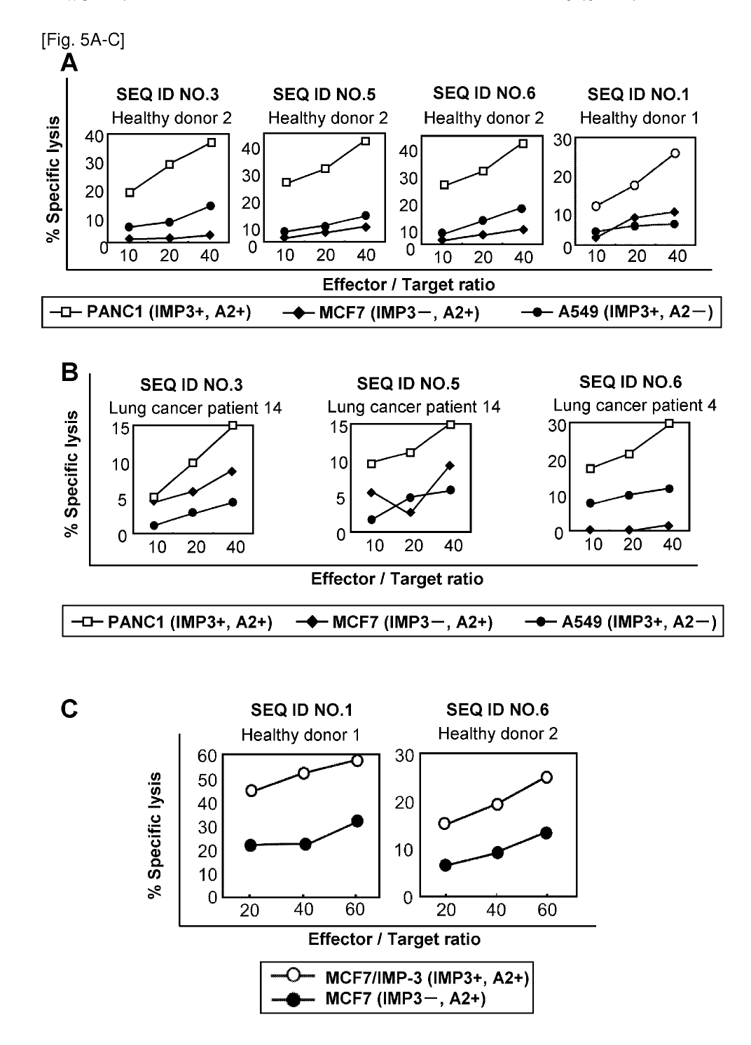

IMP-3-specific CTLs were induced from PBMCs of HLA-A2 (A*0201)-positive lung cancer patients by the stimulation with IMP-3-552-560 (SEQ ID NO: 3), IMP-3-26-34 (SEQ ID NO: 5) and IMP-3-515-523 (SEQ ID NO: 6) peptides. In FIG. 4A, the CTLs from lung cancer patients, designated patient 14 and patient 103, showed IFN-gamma production against T2 cells pulsed with IMP-3-26-34 (SEQ ID NO: 5) peptide (left panel) and IMP-3-515-523 (SEQ ID NO: 6) peptide (right panel), respectively. Compared to T2 cells pulsed with irrelevant HIV peptide, they significantly exhibited potent activity of IFN-gamma production specific to these peptides (*P<0.05). ⁵¹Cr release assay revealed that CTLs from the PBMCs of two other lung cancer patients, designated patient 4 and patient 3, showed cytotoxic activity to T2 cells pulsed with IMP-3-552-560 (SEQ ID NO: 3) peptide (left panel) and IMP-3-26-34 (SEQ ID NO: 5) peptide (right panel), without showing cytotoxic activity to T2 cells pulsed with irrelevant HIV peptide (FIG. 4B). These results indicate that these peptides induce CTLs specific to peptides not only using PBMCs of healthy donors but also using those of lung cancer patients.

Cytotoxic Activity of the CTLs Against the IMP-3 and HLA-A2 Positive Cancer Cell Line

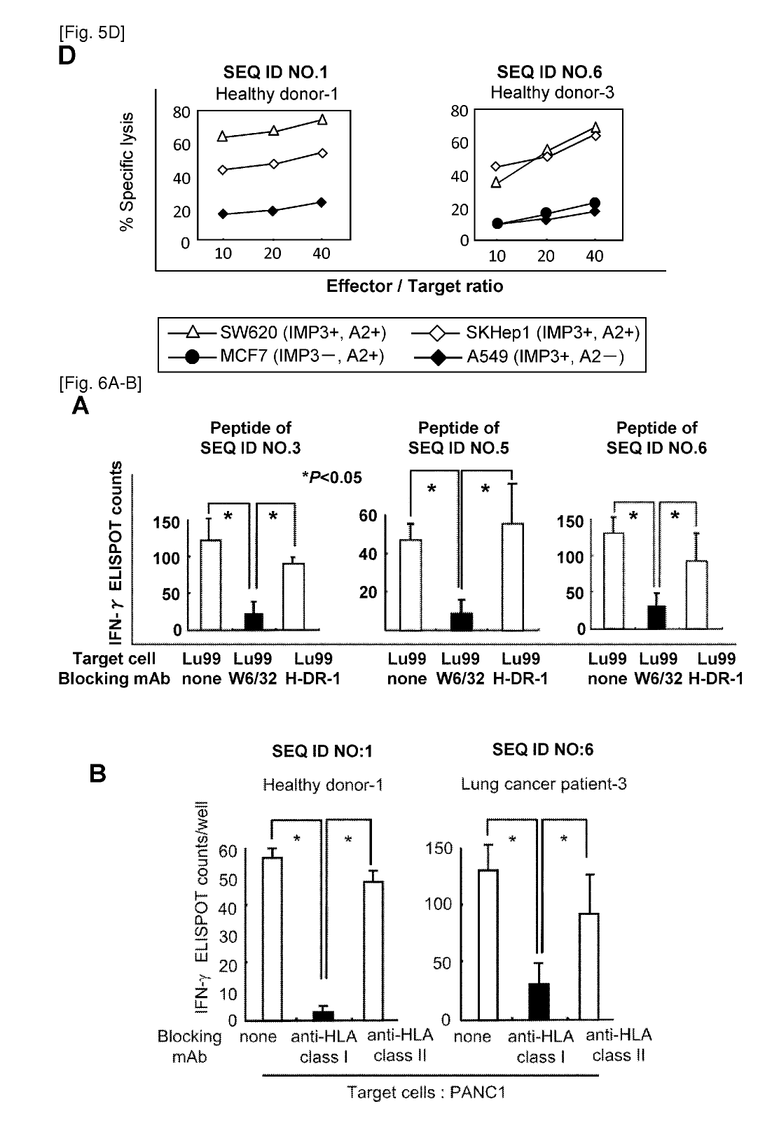

The capacity to kill human cancer cell lines expressing both IMP-3 and HLA-A2 (A*0201) was examined by ⁵¹Cr release assay. As shown in FIG. 5A, CTLs induced from PBMCs of healthy donor 2 by stimulation with IMP-3-552-560 (SEQ ID NO: 3) peptide, IMP-3-26-34 (SEQ ID NO: 5) peptide, IMP-3-515-523 (SEQ ID NO: 6) peptide and IMP-3-199-207 (SEQ ID NO: 1) showed cytotoxic activity against PANC-1, expressing both IMP-3 and HLA-A2 (A*0201). On the other hand, they showed no cytotoxic activity against MCF7, expressing HLA-A2 (A*0201) but not IMP-3, or A549, expressing IMP-3 but not HLA-A2 (A*0201). Furthermore, CTLs induced from PBMCs of the lung cancer patients, designated patient 14 and patient 4, by stimulation with the peptides having IMP-3-552-560 (SEQ ID NO: 3) peptide, IMP-3-26-34 (SEQ ID NO: 5) peptide, IMP-3-515-523 (SEQ ID NO: 6) peptide also showed cytotoxic activity against PANC-1 (IMP-3⁺, HLA-A2⁺) without showing cytotoxicity against MCF7 (IMP-3⁻, HLA-A2⁺) and A549 (IMP-3⁺, HLA-A2⁻) (FIG. 5B). The CTL lines generated from the healthy donors by stimulation with IMP-3-199-207 (SEQ ID NO: 1) or IMP-3-515-523 (SEQ ID NO: 6) peptides, exhibited cytotoxicity against MCF7/IMP-3 (MCF7 cells transfected with IMP-3 gene; HLA-A2+, IMP-3+) but not against MCF7 cells (HLA-A2+, IMP-3−) (FIG. 5C). The CTL lines generated from the healthy donors by stimulation with either IMP-3-199-207 (SEQ ID NO: 1) or IMP-3-515-523 (SEQ ID NO: 6) exhibited cytotoxic activity against SW620, SKHep1 but not against A549 (HLA-A2−, IMP-3+) or MCF7 cells (HLA-A2+, IMP-3−) (FIG. 5D).

Inhibition of CTL Responses by Anti-HLA-Class I Monoclonal Antibody

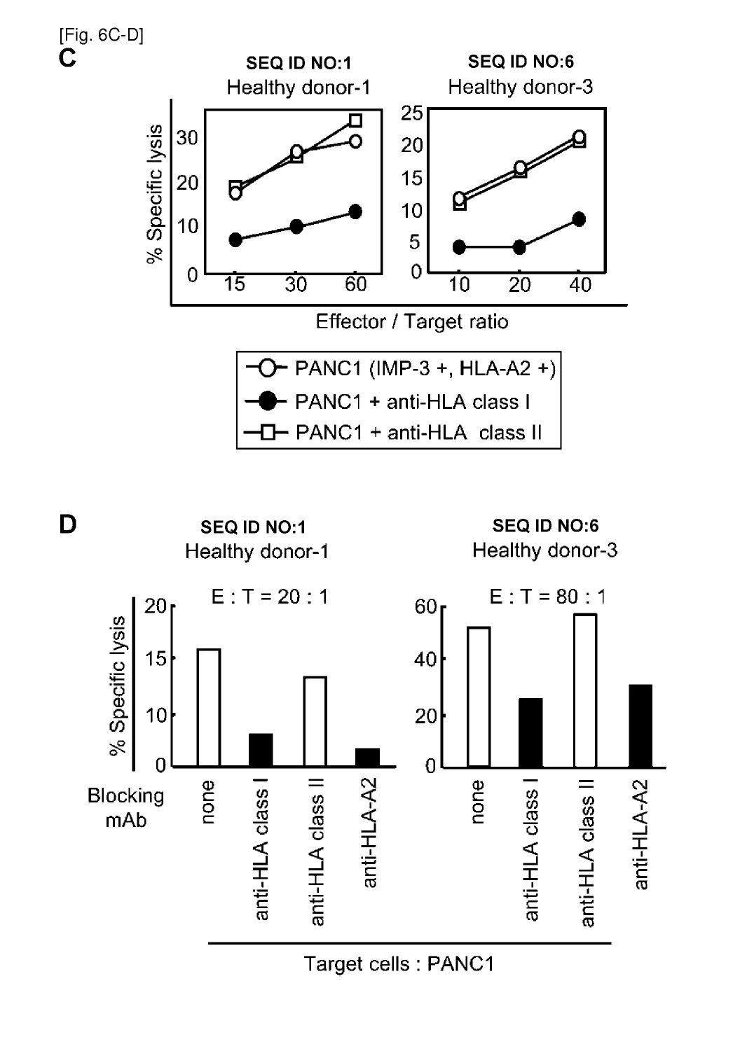

To confirm that the induced CTLs recognize the target cells in an HLA-class I-restricted manner, inhibition assay was performed using monoclonal antibody against HLA-class I (W6/32, IgG2a), HLA-DR (H-DR-1, IgG2a), anti-HLA-A2 mAb (BB7.2) to block the antigen-specific responses of the CTLs. In FIG. 6A, the inhibition of IFN-gamma production by CTLs generated from lung cancer patient 14 by stimulation with IMP-3-552-560 (SEQ ID NO: 3) peptide (left panel), IMP-3-26-34 (SEQ ID NO: 5) peptide (middle panel) or IMP-3-515-523 (SEQ ID NO: 6) peptide (right panel) was examined by IFN-gamma ELISPOT assay. The IFN-gamma production against Lu99 cells was significantly inhibited by the treatment with W6/32, but not by the treatment with H-DR-1 (* P<0.05). These results clearly indicate that these CTLs recognized target cells expressing IMP-3 in an HLA-class I-restricted manner. Furthermore IFN-gamma production and cytotoxicity were significantly inhibited by the blocking mAb against HLA-class I and HLA-A2, but not by control anti-HLA-class II mAb (FIG. 6B-D). These results clearly indicate that these peptides were naturally processed from IMP-3 protein in cancer cells and presented in the context of HLA-A2 to be recognized by peptide induced CTLs.

Homology Analysis Between the IMP3 Antigenic Peptides and Other Proteins

The CTLs stimulated with IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides showed significant and specific CTL activity. This result may be due to the fact that the sequences of IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides are homologous to peptides derived from other molecules that are known to sensitize the human immune system. To exclude this possibility, homology analyses were performed for these peptide sequences using as queries to the BLAST algorithm (http://www.ncbi.nlm.nih.gov/blast/blast.cgi) which revealed no sequence with significant homology to those peptide sequences. The results of homology analyses indicate that the sequences of IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides are unique and thus, there is little possibility, to our best knowledge, that these molecules raise unintended immunologic responses to some unrelated molecules.

In conclusion, the IMP-3-199-207 (SEQ ID NO: 1), IMP-3-552-560 (SEQ ID NO: 3), IMP-3-26-34 (SEQ ID NO: 5) and IMP-3-515-523 (SEQ ID NO: 6) peptides were identified as novel HLA-A2 (A*0201)-restricted epitope peptides derived from IMP-3 and were demonstrated to be applicable as cancer vaccines for HLA-A2 (A*0201)-positive patients with IMP-3 expressing tumors.

### INDUSTRIAL APPLICABILITY

The present invention identifies new TAAs, particularly those that induce potent and specific anti-tumor immune responses. Such TAAs warrant further development of clinical applications of peptide vaccination strategies in cancer.

All patents, patent applications, and publications cited herein are incorporated by reference.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. An isolated oligopeptide of (a) or (b) below:
(a) an isolated oligopeptide comprising an amino acid sequence selected from the group consisting of SEQ ID NOs: 1, 3, 5 and 6;
(b) an isolated oligopeptide comprising an amino acid sequence selected from the group consisting of SEQ ID NOs: 1, 3, 5 and 6, in which 1, 2, or several amino acid(s) are substituted, deleted, inserted and/or added, wherein the oligopeptide has cytotoxic T lymphocyte (CTL) inducibility.

2. (canceled)

3. The oligopeptide of claim 1, wherein the oligopeptide has one or both of the following characteristics:
(a) the second amino acid from the N-terminus is leucine or methionine, and
(b) the C-terminal amino acid is valine or leucine.

4. An isolated polynucleotide encoding the peptide of claim 1.

5. (canceled)

6. A method for inducing an antigen-presenting cell having CTL inducibility, wherein the method comprises a step selected from the group consisting of:
(a) contacting an antigen-presenting cell with the oligopeptide of claim 1, and
(b) introducing a polynucleotide encoding the oligopeptide of claim 1 into an antigen-presenting cell.

7. The method of claim 6, wherein the antigen presenting cell expresses at least one HLA-A2 antigen on its surface.

8. (canceled)

9. A method for inducing a CTL, wherein the method comprises a step selected from the group consisting of:
(a) contacting a CD8-positive T cell with an antigen-presenting cell and/or an exosome that presents a complex of the oligopeptide of claim 1 and an HLA antigen on its surface, and
(b) introducing a polynucleotide encoding a polypeptide that is capable of forming a T cell receptor (TCR) subunit binding to a complex of the oligopeptide of claim 1 and an HLA antigen on a cell surface, into a CD8-positive T cell.

10. The method of claim 9, wherein the HLA antigen is HLA-A2.

11. An isolated CTL that targets the oligopeptide of claim 1.

12. The CTL of claim 11, wherein said CTL is capable of binding to a complex of the oligopeptide of claim 1 and an HLA antigen on a cell surface.

13. The CTL of claim 12, wherein said HLA antigen is HLA-A2.

14. An isolated CTL that is induced by using the oligopeptide of claim 1.

15. The CTL of claim 14, wherein said CTL is induced by a method comprising a step selected from the group consisting of:
(a) contacting a CD8-positive T cell with an antigen-presenting cell and/or an exosome that presents a complex of said oligopeptide and an HLA antigen on its surface, and
(b) introducing a polynucleotide encoding a polypeptide that is capable of forming a T cell receptor (TCR) subunit binding to a complex of said oligopeptide and an HLA antigen on a cell surface, into a CD8-positive T cell.

16. An isolated antigen-presenting cell that presents on its surface a complex of an HLA antigen and the oligopeptide of claim 1.

17. The antigen-presenting cell of claim 16, wherein the HLA antigen is HLA-A2.

18. The antigen-presenting cell of claim 16, wherein said antigen-presenting cell is induced by a method comprising a step selected from the group consisting of:
(a) contacting an antigen-presenting cell with said oligopeptide, and
(b) introducing a polynucleotide encoding said oligopeptide into an antigen-presenting cell.

19. A method of inducing an immune response against a cancer in a subject, the method comprising the step of administering to the subject a vaccine comprising at least one active ingredient selected from the group consisting of:
(a) one or more oligopeptide(s) of claim 1, or an immunologically active fragment thereof;
(b) one or more polynucleotide(s) encoding the oligopeptide of claim 1, or an immunologically active fragment thereof;
(c) one or more isolated CTL(s) that target(s) the oligopeptide of claim 1; and
(d) one or more isolated antigen-presenting cell(s) that present(s) on its surface a complex of an HLA antigen and the oligopeptide of claim 1.

20. The method of claim 19, wherein said subject is HLA-A2 positive.

21. A pharmaceutical agent for the treatment and/or prophylaxis of cancer, and/or the prevention of a postoperative recurrence thereof, wherein the agent comprises a pharmaceutically acceptable carrier and at least one active ingredient selected from the group consisting of:
(a) one or more oligopeptide(s) of claim 1, or an immunologically active fragment thereof;
(b) one or more or a polynucleotide(s) encoding at least one oligopeptide of claim 1, or immunologically active fragment thereof;
(c) one or more antigen-presenting cell(s) presenting a complex of the oligopeptide of claim 1 and an HLA antigen on its surface; and
(d) one or more CTL(s) that is capable of binding to a complex of the oligopeptide of claim 1 and HLA antigen on a cell surface.

22. A pharmaceutical agent for inducing CTLs, wherein the agent comprises a pharmaceutically acceptable carrier and at least one active ingredient selected from the group consisting of:
(a) one or more oligopeptide(s) of claim 1, or an immunologically active fragment thereof;
(b) one or more polynucleotide(s) encoding at least one oligopeptide of claim 1, or an immunologically active fragment thereof;
(c) one or more antigen-presenting cell(s) presenting a complex of the oligopeptide of claim 1 and an HLA antigen on its surface.

23. The pharmaceutical agent of claim 21, wherein the pharmaceutical agent is formulated for the administration to a subject who is HLA-A2 positive.

24. The pharmaceutical agent of claim 21, which is a vaccine.

25-29. (canceled)
